(19) United States
(12) Patent Application Publication
(10) Pub. No.: US 2017/0173275 A1
(43) Pub. Date: Jun. 22, 2017

(54) OPTICAL SENSOR FOR NEEDLE-TIP TISSUE IDENTIFICATION AND DIAGNOSIS

(71) Applicants: Massachusetts Institute of Technology, Cambridge, MA (US); The General Hospital Corporation, Boston, MA (US)

(72) Inventors: T. Anthony Anderson, Cambridge, MA (US); Peter So, Boston, MA (US); Jeon Woong Kang, Melrose, MA (US); Ramachandra Dasari, Shererville, IN (US)

(21) Appl. No.: 15/389,770

(22) Filed: Dec. 23, 2016

Related U.S. Application Data
(63) Continuation-in-part of application No. PCT/US2015/037816, filed on Jun. 25, 2015.
(60) Provisional application No. 62/154,604, filed on Apr. 29, 2015.
(60) Provisional application No. 62/153,397, filed on Apr. 27, 2015.
(60) Provisional application No. 62/081,311, filed on Nov. 18, 2014.
(60) Provisional application No. 62/016,949, filed on Jun. 25, 2014.

(51) Int. Cl. A61M 5/42; A61B 10/02; A61M 39/10; A61M 5/32; A61M 25/06; A61B 90/30

(52) U.S. Cl. CPC A61M 5/427; A61M 25/065; A61B 90/30; A61M 39/10; A61M 5/3286; A61B 10/02; A61M 2205/3306; A61B 2090/306; A61M 2205/702; A61M 2205/52; A61M 2205/50; A61M 2210/1003; A61M 2210/1021; A61M 2210/02; A61M 2205/3313; A61M 2205/75; A61M 2205/502; A61B 5/0071

(57) ABSTRACT

The present invention relates to devices, systems and methods for spectrally identifying tissues and guiding the introduction of a probe, needle, and medical instrument into a body structure. The probe can further be used for precise delivery of therapeutic agents to selected regions of the body.

### RELATED APPLICATIONS

This application is a continuation-in-part of International Patent Application PCT/US2015/037816, filed Jun. 25, 2015, which claims priority to U.S. Provisional Patent Application No. 62/016,949 entitled “Multimodal Spectroscopy for Needle-Tip Tissue Identification” and filed on Jun. 25, 2014, U.S. Provisional Patent Application No. 62/081,311 entitled “Optical Sensor for Needle-tip Tissue Identification” and filed on Nov. 18, 2014, U.S. Provisional Patent Application No. 62/153,397 entitled “Optical Sensor for Needle-tip Tissue Identification and Diagnosis” and filed on Apr. 27, 2015, and U.S. Provisional Patent Application No. 62/154,604 entitled “Optical Sensor for Needle-tip Tissue Identification and Diagnosis” and filed Apr. 29, 2015, the entire contents of the above applications being incorporated herein by reference.

### BACKGROUND

Many surgical procedures require the introduction of needles or trocars into tissue to deliver anesthesia and other medications or to obtain tissue samples. While health care providers primarily rely on their expertise to find the right point of insertion for these instruments, they cannot differentiate between blood vessels, fat, muscle or bone once the needle or instrument has been introduced into the tissue. This blind or semi-blind approach is vulnerable to operator error, which can lead to catastrophic complications in patients including paralysis and death. Examples of procedures that use a blind or semi-blind approach for needle tip placement include epidural catheter placement, tissue biopsies, laparoscopic surgery trocar placement, joint injection, lumbar puncture, and fluid collection aspiration. Complications related to these procedures can be serious and are commonly a result of needle tip misplacement.

Each year, 2.4 million epidural catheters are placed for labor and delivery in the U.S. More than 55 million surgical cases take place in the U.S. each year, and an epidural catheter is placed for approximately 1.5 million of these procedures for post-operative pain control. Additionally, epidural corticosteroid injections are among the most common procedures completed in chronic pain clinics. Thus, significantly more than four million epidural space localization procedures take place annually in the U.S. alone. There is a high rate of obesity in surgical patients; this population is associated with a greater difficulty in placing epidural needles and catheters and, this, are at higher risk for complications. The failure rate for epidural catheter analgesia is reported to be 12-13%, and over half of these are likely secondary to the failure to accurately locate the epidural space. About 1% of epidural catheter placements (equivalent to more than 40,000 cases per year in the U.S.) lead to a postdural puncture headache (PDPH) due to the epidural needle tip passing too deep. Fortunately, spinal cord injury from this procedure is rare, but it does occur. These complications lead to significant patient morbidity as well as increased health care costs. The pain from a postdural puncture headache is severe and, without treatment, may be debilitating for several weeks or more. Serious, though rare, complications of a PDPH include neurologic symptoms and even subdural hemorrhage. The most effective treatment for a PDPH involves an epidural blood patch procedure that has additional risks and costs. Alternative treatments for PDPH include oral and intravenous caffeine, intravenous and oral fluids, and hospitalization, but are often ineffective.

A lumbar puncture is a common technique used by a variety of physicians including anesthesiologists, neurologists, emergency medicine physicians, and oncologists. This procedure is used to inject local surgical anesthesia as a diagnostic or therapeutic procedure where concern exists about meningitis, neurologic disorders, spinal cord injury, or increased intracranial pressure, and for the placement of intrathecal chemotherapeutic agents. It is difficult to ascertain the number of these procedures carried out annually in the United States. Nevertheless, approximately 25 million spinal needles are sold in the U.S. annually, and the annual lumbar puncture rate likely approaches the same number. A lumbar puncture is performed in about one percent of the 130 million emergency department visits in the U.S. annually. Complications from this procedure include cerebral herniation, injury to the spinal cord and nerve roots, hemorrhage, infection, back pain, headache, and pain or numbness along a dermatome. The incidence of a postdural puncture headache can be as high as 24-32%, which equates to six to eight million people in the U.S. each year.

Fortunately, as previously mentioned, permanent neurologic injury is a rare occurrence after neuraxial anesthesia (spinals and epidurals) but does occur. The incidence of permanent injury after spinal anesthesia ranges from 0.00-0.04% and after epidural anesthesia 0.00-0.08%. It is important to note that the cause of permanent injury is not always related to traumatic injury of the spinal cord and/or nerve roots.

Currently, epidural catheter placement is a blind, loss-of-resistance (LOR) technique that relies on sensation and clinician experience. Lumbar punctures are most commonly done in a blind, landmark fashion. The need for deep penetration and the presence of bony structures renders guidance from an ultrasound difficult for many patients, such as the obese and elderly. Previous methods have shown the ability to differentiate the epidural space from the ligamentum flavum. However, locating the ligamentum flavum is often the most difficult part of an epidural catheter placement, and the lack of information about the tissues overlying the ligamentum flavum is a major limitation of this technique.

Obesity has reached epidemic proportions in the U.S. Obesity increases the risks of general anesthesia (GA) as well as regional anesthesia (RA). Regional anesthesia is the preferred anesthetic for some surgical procedures, i.e., cesarean section. However, if RA cannot be performed because of a patient's body habitus, then a GA may be performed, further increasing the risk of complications. The authors of one study found difficulty palpating landmarks for neuraxial anesthesia in 5% of those with normal BMI, 33% of overweight patients, and 68% of obese patients. The spinous process may be greater than five centimeters from the skin and the ligamentum flavum and as deep as 12 centimeters in obese patients. In term patients, this procedure is further complicated as the optimal puncture region is smaller as the area between the spinous processes is less and the epidural space is narrower. Obese patients are more difficult to properly position and there is an increased risk of a false positive loss of resistance when attempting to find the epidural space. A study from one institution showed a 42% epidural failure rate in obese patients versus 6% in control patients. Ultrasonography (US) can be used for placing epidural catheters, typically to locate a needle insertion point and estimate the depth of the epidural space. However, in obese patients where this technique would be most helpful, ultrasonography visualization of the epidural space is more challenging and estimation of the distance from skin to the epidural space is less predictable. As image resolution and quality is inversely proportion to depth of penetration, deep structures are poorly visualized. Tracking needle movements is difficult when the needle is angled steeply such as occurs when the needle is directed toward a deep structure. Bone results in shadowing and poor or absent visualization of structures interior to it. In addition, the use of US for epidural catheter placement is typically not a real time technique. US is first used to estimate depth, and then, the loss of resistance technique is carried out.

While ultrasonography can be used for epidural needle and catheter placement, it does not decrease the number of attempts in patients with easily palpable spinous processes and may increase total procedure time. The use of ultrasound by anesthesiologists experienced with both ultrasound and landmark techniques does not increase the success of spinal anesthesia or reduce complications, traumatic puncture rates, or number of attempts, and using ultrasound may decrease procedure time by a small amount or not at all. Other studies have found a decreased number of procedure attempts and failed and traumatic neuraxial blockade with the use of pre-spinal puncture or pre-epidural needle ultrasonography to assess for an ideal needle insertion point as well as tissue depth. The authors of one study concluded that pre-procedural ultrasound scanning should be limited to technically challenging patients only, and it has been suggested that ultrasound use may not improve outcomes for those with normal anatomy and Body Mass Index (BMI). False positives occur with the use of ultrasound for locating the epidural space, and these cases can be very dangerous by posing an increased risk of neuraxial injury.

Peripheral nerve blocks are a type of regional anesthesia that relies upon accurate placement of needles and catheters. Local anesthetic is injected near a specific nerve or bundle of nerves to block painful sensations from a specific area of the body. Nerve blocks usually last longer than the simple injection of local anesthesia around a painful site. Ultrasound guided nerve blocks are the most common technique currently used, but the location of the needle tip relative to the nerve or bundle of nerves cannot always be accurately assessed. Of patients who receive a peripheral nerve block, one to two percent have residual sensory or motor deficit after the effect of the local anesthetic wears off. This is usually temporary (days to months) and rarely permanent. It is believed that these deficits are in part due to damage from the needle and/or injection of local anesthetic too close to the nerves (within the nerve bundle instead of just outside of it).

Among many different tissue biopsy procedures, there are one million prostate biopsies performed annually in the U.S. Prostate biopsies to diagnose and determine cancer staging are carried out trans-rectally and involve multiple biopsy needle passes to sample adequate tissue in order to locate potential cancer sites within the prostate. Complications from prostate biopsies include bleeding, infection, and pain. Post-biopsy infection rates are as high as 6%. Post-biopsy hospital admission rates are 1-4%, most due to infections. Infections arise from translocation of bacteria from the rectum into the prostate, bloodstream, and bladder. If fewer samples were required during the biopsies, the risk of infection would decrease. In addition, a 25% reduction in infection related inpatient admission would save an estimated $120 million annually in the U.S. alone.

In the U.S., more than two million laparoscopic surgeries are performed annually. A standard laparoscopic technique uses a spring-loaded Veress needle to create pneumoperitoneum for laparoscopic surgery. A spring-loaded blunt stylette lies inside the needle sleeve and extends beyond the needle tip. The stylette retracts as the needle is pushed through abdominal tissue and automatically advances forward once the peritoneum is penetrated. Laparoscopic techniques have revolutionized modern surgery and lead to decreased postoperative pain, faster recovery, and fewer overall complications. However, this type of surgery is associated with unique complications such as inadvertent bowel, abdominal organ, or vascular injury that may occur while gaining access to the abdomen. One survey reported that the average incidence rate of trocar-related bowel injury is less than 1% while the rate of trocar-related vascular injury is 0.1%. The rate of complications from port site trocar insertion is about 0.2%. A 1996 study found that 83% of vascular injuries, 75% of bowel injuries, and 50% of local hemorrhage injuries occur during the primary port entry as it is a blind technique. Complication rates for trocar-related vascular and bowel injuries can be especially serious because detection of such injuries is often delayed or absent at the time of the surgery. The inadvertent placement of the initial needle for laparoscopic surgery into a major blood vessel or organ can be catastrophic as subsequent insufflation with CO₂ gas may result in hemorrhage, brain damage, or death. To minimize entry-related injuries, several techniques, instruments, and approaches have been introduced during the last century. These include the Veress-pneumoperitoneum-trocar, “classic” or closed entry, the open (Hasson) technique, direct trocar insertion without prior pneumoperitoneum, the use of shielded disposable trocars, the optical Veress needle, optical trocars, radially-expanding trocars, and a trocar-less reusable, visual-access cannula. Each of these methods of entry enjoys a certain degree of popularity according to the surgeon's training, experience, and bias, and according to regional and interdisciplinary variability. Unfortunately, most if not all of these techniques have failed to produce significantly reduced injury rates.

Needles and endoscopes may be placed in arthroscopic procedures to address medical issues in and around a joint. For example, patients with worn cartilage may develop bone spurs in a joint that can break down joint fluid. Injections of treatments containing hyaluronic acid can alleviate these symptoms, but the injection must be placed correctly in a location where it is needed.

Needle guidance technology has applications for improving the safety of cancer biopsy procedures, such as those of the prostate. Prostate cancer is the second leading cause of cancer death in men in the United States. According to American Cancer Society statistics, about 233,000 new cases of prostate cancer will be diagnosed in 2014 with an expected annual mortality of 29,480. While prostate cancer is typically detected by digital rectal examination and blood test screening for prostate-specific antigen, the definitive diagnosis and grading of prostate cancer often requires needle biopsy. Typically, a biopsy involves the use of 18 gauge needles to remove 8-18 tissue samples guided by transrectal ultrasonography to locate the gland. Therefore, similar to epidural needle placement, there are serious consequences to needle misplacement. As it is one of the most common tumor biopsy procedures with over one million performed per year in each of the U.S. and Europe, reducing biopsy-related complications has a significant socioeconomic benefit. Bleeding from the urethra or rectum is very common though often minor. Approximately 3% of patients who receive a prostate biopsy will require follow-up due to bleeding complications. In addition, post-prostate biopsy infection rates are as high as 6%. Currently, no reliable device exists to reduce these complications. The American Cancer Society reports 74,690 new cases of bladder cancer per year in the U.S. with an expected annual mortality of 15,580. The risk of bladder cancer biopsy is similar to that of prostate cancer with complications include bleeding and bladder perforation. One study reported a bladder perforation rate of up to 9%; this outcome requires increased medical care and a longer hospital stay.

Many biopsy procedures (e.g., those for prostate cancer) rely on random sampling. Breast cancer is the most common disease for women in the U.S. The American Cancer Society predicts 232,670 new cases of invasive breast cancer in 2014 with an expected annual mortality of 40,000. While most breast cancer biopsies are guided by x-ray imaging, the deformability of breast tissue can result in misplacement of the needle during a biopsy procedure resulting in tissue removal from the wrong location and the need to repeat the entire procedure. Previous research has demonstrated the use of needle tip spectroscopy measurements to guide a needle directly to the correct biopsy site based on the presence of microcalcifications.

It should be noted that these two applications have very different levels of difficulty. Currently, needle biopsies are often performed by landmark and palpation techniques alone. If an additional method could be used to aid with needle placement, clinicians could be more certain that the tip of the needle is in the appropriate tissue space prior to initiating the medical procedure. While ultrasound can be used to aid with some cases, it is of limited utility if the tissue of interest is deep or if bone blocks the ultrasound signal. There is a tremendous need for a simple, low-cost guidance technology for needle placement that can be readily integrated into existing clinical workflow. Specifically, there is a clinical need for a device that allows rapid identification of tissue type at the tip of a needle to ensure that the needle is inserted in the correct tissue space.

### SUMMARY

The invention relates to a spectroscopic sensor system to identify tissue during needle insertion. Preferred embodiments use a light source, fiber optic delivery and collection through a needle, a light detection system, a memory device, and a data processor to process spectral data at points during insertion. Spectra are obtained and compared to preselected spectral profiles stored with the memory device to determine needle-tip location. Preferred embodiments utilize Raman spectral, intrinsic fluorescence, and/or diffuse reflectance measurements to measure a sequence of tissue types that are frequently situated in adjacent layers. For certain applications, a needle used for entry through these layers can cause the intrusion of body fluids, such as blood, into the field of view of the probe, thereby complicating the measurement. Consequently, in certain embodiments, methods can be used to enhance the accuracy of the measurement which account for the presence of such fluids.

Using needle tip spectroscopy to guide a biopsy, for example, is difficult as it requires the detection of the lesion's presence within a normal tissue environment where the volume fraction of the lesion may be very small and the spectroscopic signature differences may be much more subtle. Multi-modal spectroscopy (MMS) using Raman spectroscopy (RS), diffuse reflectance spectroscopy (DRS), and intrinsic fluorescence spectroscopy (IFS) can measure biochemical and morphological information about tissues non-destructively. MMS has previously been shown to differentiate between cancerous and normal tissues and to enable identification of atherosclerotic plaques, for example.

A preferred embodiment is an optical sensor that fits inside existing surgical instruments and needles to enable operator differentiation between blood vessels, fat, muscle or bone, for example. This allows users of these instruments to efficiently deliver medications and obtain tissue samples without the risk of accidentally puncturing other tissues.

A preferred embodiment of the present invention relates to multimodal spectroscopy (MMS) as a clinical tool for the in vivo diagnosis of disease in humans. The MMS technology can combine Raman and fluorescence spectroscopy for example. A preferred embodiment involves diagnosis of cancer of the breast and of vulnerable atherosclerotic plaque, esophageal, colon, cervical and bladder cancer. MMS is used to provide a more comprehensive picture of the metabolic, biochemical and morphological state of a tissue than that afforded by either Raman or fluorescence and reflectance spectroscopies alone.

In preferred embodiments, RS is used as a singular diagnostic modality such as where RS can differentiate the tissues overlying the epidural space (e.g., skin, fat, muscle, supra-/intra-spinous ligament, and ligamentum flavum) and those beyond it (e.g., dura mater and spinal cord) in an ex vivo animal model. RS can also differentiate tissues of the abdominal wall and abdomen (i.e., skin, fat, muscle, liver, spleen, pancreas, and kidney).

Preferred embodiments can also include a needle with an integrated miniaturized MMS probe, which can identify tissues through which it passes, targeting the application of epidural catheter placement. The system operates to identify the tissues overlying the spinal cord through which it passes. The system may differentiate tissues within and around joints, abdominal wall components from abdominal organs, and normal from abnormal tissue during biopsies and fluid collection aspiration.

In a preferred system, a needle for measuring tissue includes a fiber optic probe having a diameter of 2 mm or less. This small diameter allows the system to be used for structure identification and diagnosis of diseases in small lumens or soft tissue with minimal trauma. A delivery optical fiber is included in the probe coupled at the proximal end to a light source. A filter for the delivery fibers is included at the distal end. The system includes a collection optical fiber (or fibers) in the probe that collect scattered Raman radiation from tissue; the collection optical fiber is coupled at the proximal end to a detector. A second filter can be disposed at the distal end of the collection fiber(s). An optical lens system is disposed at the distal end of the probe including a delivery waveguide or filter coupled to the delivery fiber; a collection waveguide or filter can be positioned between the collection fiber and a lens. In some embodiments, the first and second filters may be applied directly to the distal ends of the delivery and collection fiber(s) using, for example but not limited to, chemical or physical vapor deposition.

The invention is an optical sensor coupled to a detector that can distinguish between different types of tissues and diagnose disease status of tissues by applying spectroscopic methods. The sensor can be customized to accommodate different applications in which a probe with a small enough diameter can be inserted into invasive medical instruments and surgical needles. The sensor system includes a needle, preferably a metal needle, to protect the optical sensor from the environment, one or more collection fibers with long-pass filters to collect inelastically backscattered light from the tissue, and a sapphire ball lens to focus excitation light onto a small tissue spot and effectively collect back-scattered light into the collection fibers. A metal sleeve can be used to minimize the cross-talk between lights in the delivery and collection fibers. One or more excitation fibers may be provided with laser line filters to deliver excitation light without generating background signal in the collection fibers, and a sapphire window may be used to maximize the collection efficiency. A light source and detector system are optically coupled to the delivery and collection fibers, respectively.

A preferred embodiment involves applications in which the optical sensor is positioned within a shaped needle such as a Tuohy shaped needle, which is commonly used in epidural catheter placements. This allows the user to identify different types of tissues before inserting the needle into the target site for epidural catheter placement. Due to its small diameter (e.g., 17 gauge), the sensor can also enable the passage of saline through the needle in order to confirm a positive target site using the loss-of-resistance method (LOR), which is currently standard practice for this procedure. Tissue identification combined with LOR confirmation is expected to significantly reduce patient complication rates from improper needle insertion or the insertion of the needle into the wrong tissue.

Additional embodiments can include applications for this device such as guidance in spinal/lumbar puncture injections, joint injections, tissue biopsies, fluid collection localization and aspiration, surgical instrument and trocar placement, peripheral nerve blocks, and epidural steroid injections. The invention can include embodiments in which a probe can fit as an insert in the applicable procedural instrument.

A preferred embodiment includes an epidural needle that can safely navigate the tissues overlying the spinal cord using Raman or multimodal spectroscopy (MMS) to identify the tissues through which it passes in addition to the use of the traditional LOR technique. Epidural needle placement and the use of multimodal spectroscopy for accurate needle tip tissue identification are employed. This device has numerous applications. Further potential uses for this needle and optical system include surgical trocar placement at the start of laparoscopic surgeries in order to verify correct placement of the initial needle in the abdomen. The inadvertent placement of the initial needle for laparoscopic surgery into a major blood vessel or organ can be catastrophic. Thus, the present application is directed to spectroscopic devices and methods that differentiate between the tissues of the abdominal wall and abdominal organs. Other embodiments are directed to devices for tissue needle biopsy, peripheral nerve blockade during and after surgery, orthopedic joint injection, and drainage of fluid collections in the abdomen and thorax. As described above, a needle MMS system can be used for epidural catheter placement, in vivo tissue needle biopsies, peripheral nerve blockade, joint injection/aspiration, and in vivo drainage of fluid collections.

An exemplary embodiment provides a spectroscopy-based clinical device with a miniaturized MMS probe that can be used to provide rapid and accurate real time identification and discrimination between biological tissues. The device is used to guide clinicians and has applications for a diverse range of surgical procedures.

One of the critical components for needle-tip tissue identification is an ultra-thin Raman probe. A Raman spectroscopy probe with less than 1 mm diameter is used for epidural catheter placement. There are other needle placement procedures that will also require further miniaturization. The system uses an ultra-thin RS probe, needle, and clinically applicable spectroscopy instrument. The epidural needle equipped with an ultra-thin RS probe can be inserted into a patient that is under general anesthesia. Spectroscopic signals from different epidural needle trajectories can be utilized.

The key challenge in adapting multimodal spectroscopy for needle-based procedures is the development of a miniaturized fiber probe. Several generations of fiber optic probes have been developed for in vivo use. There are two challenges in the design of a smaller probe. The first hurdle originates from the inherently weak Raman signals in vivo. Approximately 1 of every 10⁶ excitation photons that reach the tissue will be converted into a Raman photon. The second hurdle lies in the optical characteristics of the tissue. The signal of interest is directly attenuated by tissue absorbance of the excitation laser light and the signal photons. Furthermore, signal collection is confounded by light scattering, which causes the photons to be widely diffused over large areas and angles. Therefore, it is critical to optimize throughput and maximize collection efficiency to achieve a sufficient signal-to-noise ratio (SNR) for accurate analysis within a clinically realistic time frame. Because of these difficulties and the severe size constraints, micro-optical design is challenging and requires understanding the optical properties of the target sample. Various embodiments describe an optical fiber probe that has been optimized for multimodal spectroscopy through the removal of a majority of the optical fiber background and the placement of appropriate bandpass filters at the distal ends of the excitation and collection fibers to optimize detection of weak, especially Raman, signals from tissues. The collection of scattered tissue signals is maximized by optimizing placement of excitation fibers and collection fibers and by designing a custom microspherical front end lens. In several embodiments, the probe design has a rigid distal tip of less than a few millimeters in length and 2 mm in diameter and is able to collect tissue spectra in 1 second. In several embodiments, the probe design is front viewing; therefore, the probe can detect the transition between tissue types in the path of the needle before puncturing the next tissue layer.

Although others have developed fiber-optic based spectroscopic probes for various applications, their sizes are still significantly larger than most clinical needles. Currently, there are no commercially available Raman probes less than 2 mm in diameter with a ball lens and less than 1 mm in diameter without a ball lens. A ball lens focuses the excitation beam onto a small area of tissue and effectively collects back-scattered light into collection fibers. While Raman probes with these form factors have been successfully applied to many applications (i.e., guiding breast core needle biopsies and detecting atherosclerosis), they are still too large for insertion into small, clinically-used procedure needles.

For example, the inner diameter of a 17-gauge Tuohy epidural needle is 1.15-1.19 mm. Therefore, it is essential to design and fabricate a new, thinner probe for identifying the tissue at the needle tip. In order to determine the most effective spectroscopy method for epidural needle placement, we applied DRS, IFS and Raman spectroscopy to dissected swine tissues from skin to spinal cord. While DRS and IFS showed meaningful differences, Raman spectroscopy showed the best differentiation of each tissue. An embodiment of the present application comprises a Raman probe of less than 1 mm diameter which fits into an epidural needle (17-gauge Tuohy needle).

### BRIEF DESCRIPTION OF THE DRAWINGS

FIG. 1 illustrates a needle guidance system in accordance with a preferred embodiment of the invention;

FIG. 2 shows the placement of a surgical puncture device in the epidural space of a patient according to various embodiments;

FIGS. 3A-3P show side and end views of exemplary embodiments of a probe device;

FIG. 3Q shows a side view of a needle illustrating detection zones according to various embodiments;

FIGS. 3R-3U show side and end views of exemplary embodiments of a probe device;

FIGS. 4A-4D illustrate cross-sectional side and end views of tissue probes according to various embodiments of the invention;

FIG. 5 schematically illustrates a multimodal system according to various preferred embodiments;

FIG. 6 illustrates spectra acquired from dissected and isolated tissues using a tissue probe as described in various embodiments;

FIG. 7 illustrates spectra acquired from different tissues during a single puncture event using the tissue probe as described in various embodiments;

FIG. 8 illustrates microscopic views of various stained tissues alongside corresponding Raman spectra obtained from dissected tissue and corresponding Raman spectra obtained at a needle-tip during needle insertion;

FIGS. 9A and 9B display the four basis Raman spectra and a representative fitting result, respectively;

FIG. 10 displays the fitting coefficients representing the decomposition of each tissue layer's Raman spectrum into the four basis spectra;

FIG. 11 displays a decision tree based on the four coefficient fitting method;

FIG. 12A is a longitudinal view of an apparatus including a probe for measuring tissue in accordance with a preferred embodiment of the present invention;

FIG. 12B is a transverse view of the probe illustrated in FIG. 12A in accordance with a preferred embodiment of the present invention;

FIG. 12C is a longitudinal view of an apparatus including a probe for measuring tissue in accordance with an embodiment of the present invention;

FIGS. 13A-13E show views of a preferred embodiment of a probe and needle for measuring tissue in accordance with the present invention;

FIG. 14 illustrates a longitudinal view of a preferred embodiment of a side-viewing probe for measuring tissue in accordance with a system of the present invention;

FIGS. 15A and 15B are end views of an MMS probe and a RS probe in accordance with the invention;

FIG. 16 is a side cross-sectional view of a side looking probe;

FIG. 17A is a forward looking MMS probe with a ball lens;

FIG. 17B is a forward looking MMS probe with a half ball lens;

FIG. 18 is a schematic diagram illustrating a system for measuring tissue in accordance with a preferred embodiment of the present invention.

FIG. 19 is a schematic of an MMS system;

FIG. 20 is another embodiment of an MMS system in which a probe can be used in a scanning system;

FIG. 21 is a schematic diagram of a method of performing a guided needle insertion procedure in accordance with preferred embodiment of the invention;

FIG. 22 illustrates spectra acquired from tissue and other material located in and around a joint using a system according to various embodiments;

FIG. 23 illustrates spectra acquired from tissue and other material in and around an abdominal region with a system according to various embodiments;

FIG. 24 illustrates a process sequence for processing and displaying needle guidance data.

### DETAILED DESCRIPTIONS OF PREFERRED EMBODIMENTS

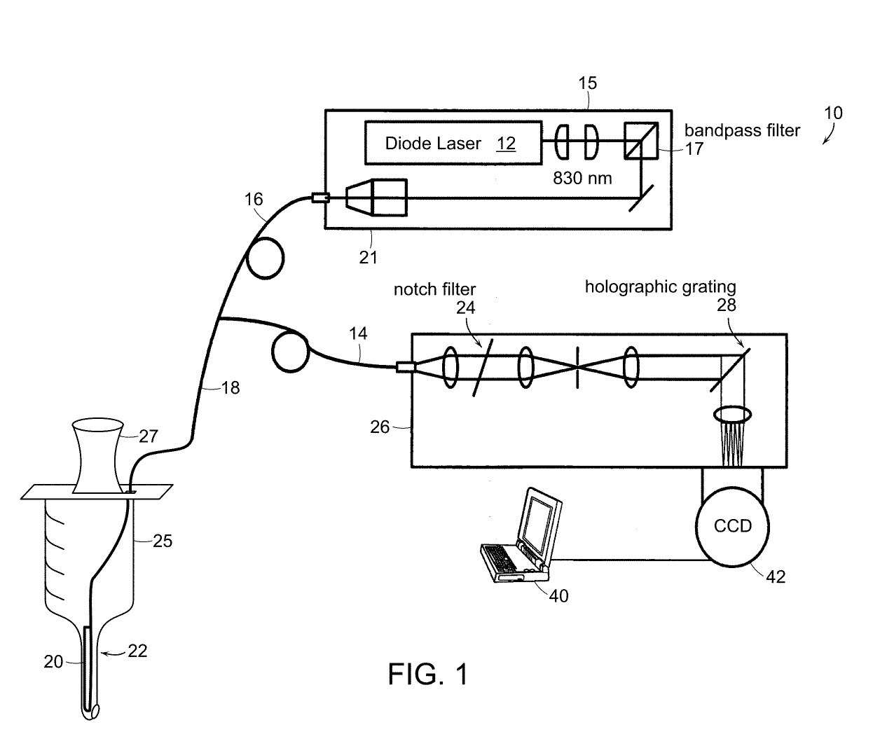

Shown schematically in FIG. 1 is a schematic diagram illustrating a needle guidance system 10 in accordance with preferred embodiments of the invention. A light source system 21 is coupled to one or more delivery optical fibers 16 which delivers excitation light through probe 20 onto a tissue region to be measured. The probe 20 is positioned within a needle 22 during the measurement. The needle 22 can be associated with a disposable syringe 25 having a plunger element 27 actuated by a user to deliver a therapeutic agent and/or conduct a loss-of-resistance procedure. The light source system 21 may use a range of illumination sources 12 depending upon the desired application. Illumination sources 12 include, but are not limited to, broadband lamps, narrow-line lamps, and a range of laser sources (e.g., gas, solid-state, dye, or diode lasers). In a preferred embodiment, an illumination source 12 emitting at a wavelength longer than 750 nm, such as an argon-pumped Ti:sapphire laser system or a diode laser is used. The diode laser may be an InGaAs laser emitting at 785 nm or 830 nm, such as, for example, fabricated by Process Instruments, Salt Lake City, Utah. The laser output is band pass-filtered 17 and is coupled into the delivery optical fibers 16 which are connected to the probe 20. The light source system 21 may comprise a number of optical elements including lenses 15, beamsplitters, and mirrors. The light is incident on the tissue, and Raman-scattered light from the tissue is collected by one or more collection optical fibers 14. The collection optical fiber(s) 14 couple light from the tissue region to a spectral detection system 26. In the spectral detection system 26, the light may be notch-filtered 24 and projected onto an entrance slot of a spectrophotometer. The notch filter removes Rayleigh-scattered laser light. Inside the spectrograph, a grating disperses light onto a CCD detector 42. The CCD interface and data storage and processing is provided in a computer 40 such as a personal computer. A program such as Winspec Software provided by Princeton Instruments can be used to interface the CCD 42 with the personal computer 40, which performs the data processing and storage functions. In alternate embodiments, the Labview program by National Instruments, Austin, Tex., is used to interface the CCD 42 with the personal computer 40. Raman signals are read from the CCD 42, collected by the computer 40 and stored on a computer readable media for later analysis or may be used for real time analysis in a clinical setting.

A multi-modal spectroscopy (MMS) system combines diffuse reflectance spectroscopy (DRS), intrinsic fluorescence spectroscopy (IFS), and Raman spectroscopy (RS) and can measure biochemical and morphological information about tissues non-destructively through an optical fiber probe. These spectroscopic techniques have previously been shown to distinguish margins between cancerous and normal breast tissue and to enable identification of vulnerable atherosclerotic plaques. Furthermore, these methods have been used to improve breast biopsy success rates and to classify lesions in real time. While spectroscopy has been effective in harvesting better breast lesion samples, the biopsy needle diameter is relatively large. MMS technology applied to needle placement applications, such as epidural catheter placement, requires further miniaturization. When combined with current techniques, these spectroscopic methods have the potential to reduce the rate of procedural complications due to needle tip misplacement as well as costs related to procedural time.

Multimodal spectroscopy is a contact probe spectroscopic technique for real time cancer detection. Tri-modal spectroscopy was used in the non-invasive diagnosis of epithelial dysplasia based on combining three spectroscopic modalities: diffuse reflectance (DRS), intrinsic fluorescence (IFS), and light-scattering spectroscopy (LSS). Subsequently, this approach was refined into multimodal spectroscopy by combining DRS, IFS, and Raman spectroscopy (RS). Each modality provides complimentary physical and biochemical tissue parameters that can be used to diagnosis the disease state of tissues.

DRS measures the spectrum of near-UV-visible light (300 to 700 nm) traversing turbid biological tissue. The resulting spectrum exhibits distinct features due to scattering and absorption of the incident light by the tissue. The present invention uses a model to analyze DRS spectra based on an analytical expression for diffusion of multiply-scattered light to extract the wavelength-dependent coefficients of scattering and absorption. For example, in our study of atherosclerotic plaque, key absorbers are hemoglobin and beta-carotene. Fluorescence spectroscopy relies on the excitation of molecular electronic energy levels, which gives rise to re-emission at wavelengths longer than that of the excitation light. The emission spectrum provides information about the fluorophores excited. The primary fluorophores in tissues include collagen, NADH, elastin, and tryptophan. RS detects molecules by exciting vibrations among bonds that are unique to each molecule and has been used extensively in biomedicine. Previous work by our group on RS of atherosclerosis identified eight key morphological components in arterial pathogenesis that could be identified by their Raman spectral signatures and include collagen fibers, cholesterol crystals, calcium mineralization, and foam cells. Multimodal spectroscopy has been successfully applied in the diagnosis of cancer in the oral cavity, uterine cervix and esophagus. In each organ, multimodal spectroscopy diagnosed diseases with high sensitivity and specificity.

Diffuse reflectance spectroscopy (DRS) is based on white light absorption. Tissue is illuminated by a white light source from an excitation fiber. Backscattered signal from tissue is collected by collection fiber(s). Using numerical modeling, absorption as well as scattering parameters can be calculated. Absorbers from human tissue include oxy-/deoxy-hemoglobin, β-carotene, and melanin. However, DRS analysis heavily depends on the modeling, which is very sensitive to measurement geometry. Specifically, modeling assumes a constant probe-tissue distance. Most DRS measurements are performed using a contact probe. If this assumption is not met, DRS calculations are not valid. This point is critical for needle-tip measurements because procedural needles must be moved in order to locate the correct injection site and these movements will not guarantee probe-tissue contact.

Intrinsic fluorescence spectroscopy (IFS) is based on ultra-violet (UV) induced fluorescence. Tissue is illuminated by an UV laser from an excitation fiber. Backscattered fluorescence signal from tissue is collected by collection fiber(s). Acquired fluorescence spectra are affected by tissue absorption. Pure fluorescence spectra can be calculated in combination with DRS. Collagen, elastin, keratin, NADH, and FAD generate strong UV-induced fluorescence signals. The advantage of IFS is strong signal from connective tissues. However, IFS requires DRS for analysis, and fluorescence signals from tissues are not stable due to photobleaching (destruction of a fluorophore by an excitation light exposure). Moreover, this technique does not differentiate non-connective tissues. For example, IFS cannot differentiate muscles from fatty tissues.

Raman spectroscopy (RS) is based on inelastic light scattering. Near-infrared (NIR) lasers are widely used for tissue Raman spectroscopy due to their safety and superior background performance. Excitation light is delivered by a fiber and a portion is inelastically scattered from tissue. This Raman scattered light is collected by collection fiber(s) and contains chemical information from the sample. Raman spectroscopy is based on the interaction between excitation light and chemical bonding of a sample and, as such, it delineates the chemical composition of the tissue. Most important biomolecules have their own specific Raman signal. Raman spectroscopy can clearly distinguish protein, fat, and connective tissues.

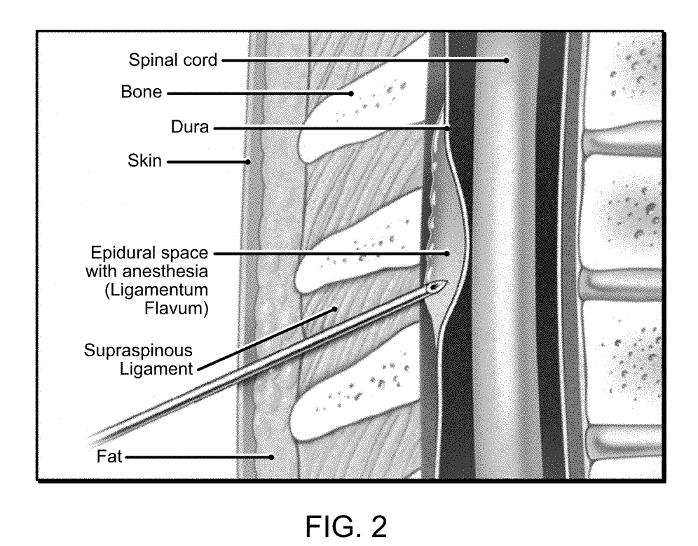

RS can differentiate the tissues overlying the epidural space shown in FIG. 2 (e.g., skin, fat, muscle, supra-/intra-spinous ligament, and ligamentum flavum) and those beyond it (e.g., dura mater and spinal cord) in an ex vivo animal model. In addition, RS can differentiate tissues of the abdominal wall (e.g., skin, fat, and muscle) from abdominal organs (e.g., liver, spleen, and kidney) in the same model.

Raman spectroscopy can distinguish each tissue type from skin to spinal cord in an ex vivo porcine model. Real-time RS of intact porcine paravertebral tissues along both the midline and paramedian trajectories using a probe-in-needle device confirmed the individual tissue results and allowed identification of each tissue the needle tip was adjacent to. Thus, it is possible to use RS to improve epidural needle, epidural catheter, and spinal needle placement by reducing procedure time and complication rates while improving success rates. A Raman spectroscopy probe can be adapted to fit inside a standard 17-gauge Tuohy needle and provide spectroscopic guidance while still using the standard loss of resistance technique. In an exemplary embodiment, an RS probe is fit into a 17-gauge epidural needle and allows the simultaneous use of RS and the LOR technique for detection of the epidural space.

Fiber-optic spectroscopic probes for atherosclerosis detection, cancer diagnosis, and biopsy guidance can be used for needle guidance can be used for needle guidance. In a preferred embodiment, the optical instrument can perform diffuse reflectance, intrinsic fluorescence, and Raman spectroscopy simultaneously with a single fiber-optic probe. This multi-modal probe guarantees co-registration among different spectroscopy modalities. In various embodiments, the probe includes one central excitation fiber (200 μm core diameter, 0.22 NA) optically isolated from the surrounding 15 collection fibers, ten of which (200 μm core diameter, 0.26 NA) collect Raman spectra and five of which (200 μm core diameter, 0.22 NA) collect DRS and IFS light. The probe tip contains a module to filter the excitation and collection light and a sapphire ball lens to optimize collection. The MMS probe (with a focusing ball lens) diameter is 2 mm.

The current size-limiting factor is the optical filters at the tip of fiber probes. Generally, fiber-optics based Raman probes include two types of filters. One is in the excitation beam path and the other is in the collection path. In the excitation beam path, either a laser band-pass filter or a short pass filter is attached at the tip of the excitation fiber. The role of these filters is to reduce the background signal, which is mostly generated from the delivery fiber. By filtering out light of longer wavelength than the excitation wavelength, the collected Stokes Raman spectrum can be isolated from the fiber background. The second filter is attached at the tips of the collection fibers. Either a laser notch filter or a long pass filter is used to reject the back-scattered Rayleigh light (at the excitation wavelength). Since Raman scattering is a weak process, it is important to reject elastically scattered light from the collected signal. Otherwise, the back-scattered Rayleigh light will generate fluorescence background signal from the collection fibers. In an exemplary embodiment, the fabrication process includes depositions of multilayer coatings on top of micro disks (excitation) and micro tubes (collection) in a custom coating chamber. These filters are manually glued to the fiber tips under the microscope by experienced engineers. Since these Raman probes are assembled manually, their production is labor intensive and slow. More importantly, a relatively large cross section area of the probe is not used for measurement. For example, the latest MMS probe utilizes only 20% of the probe area for light collection.

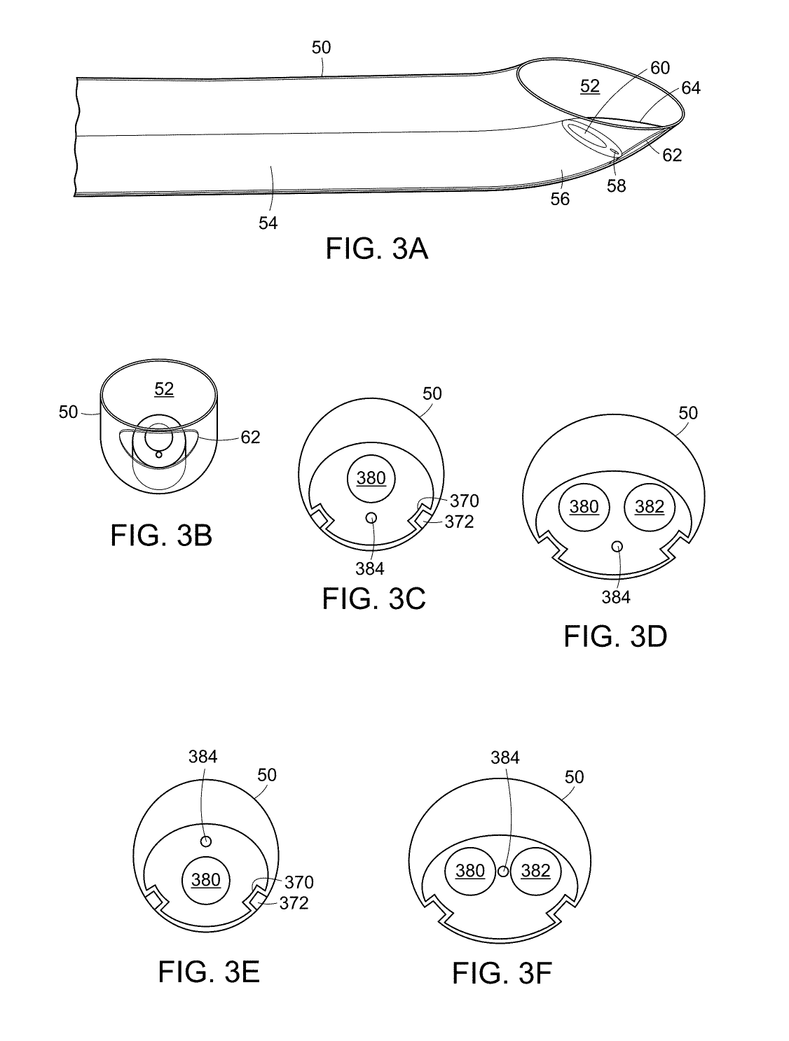

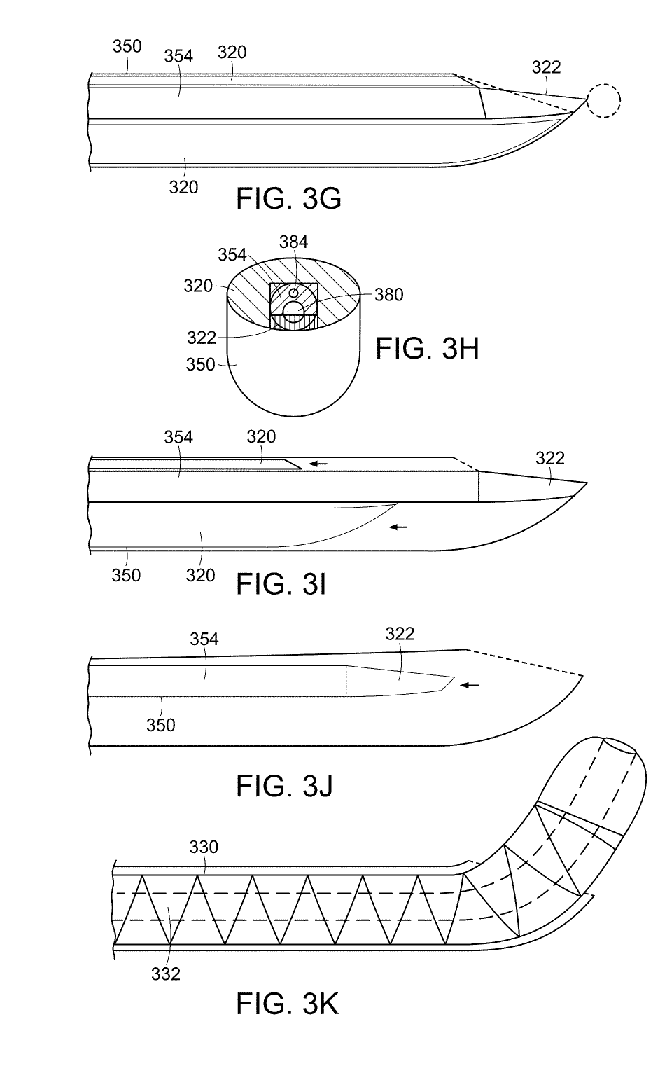

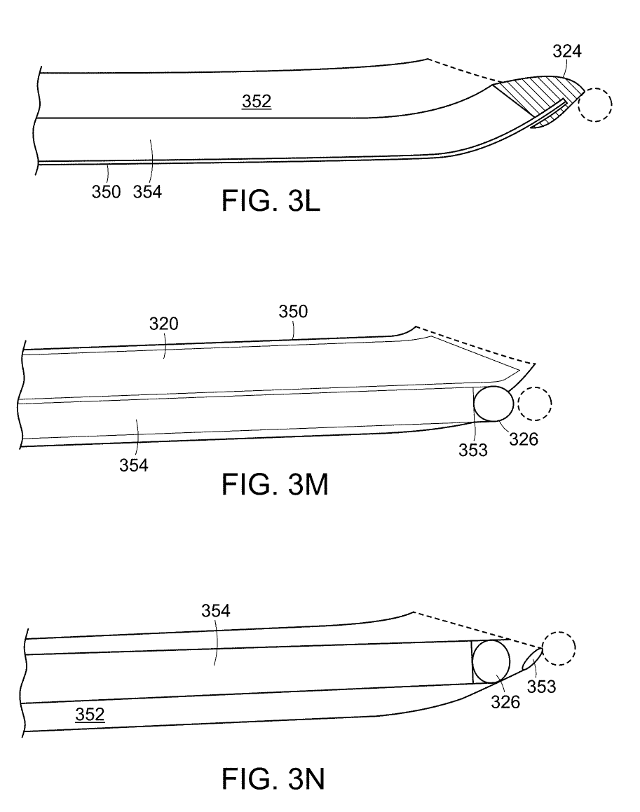

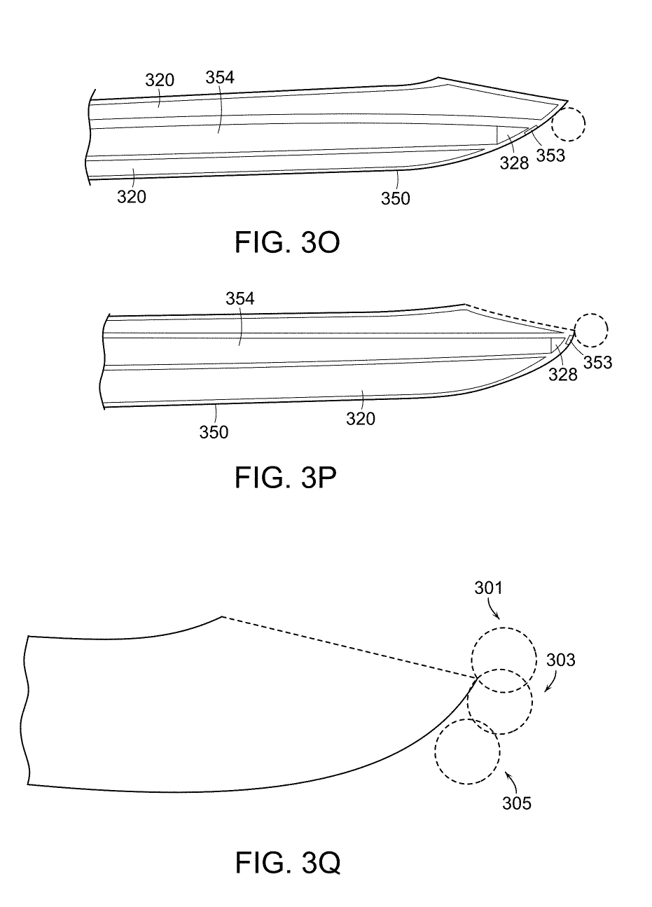

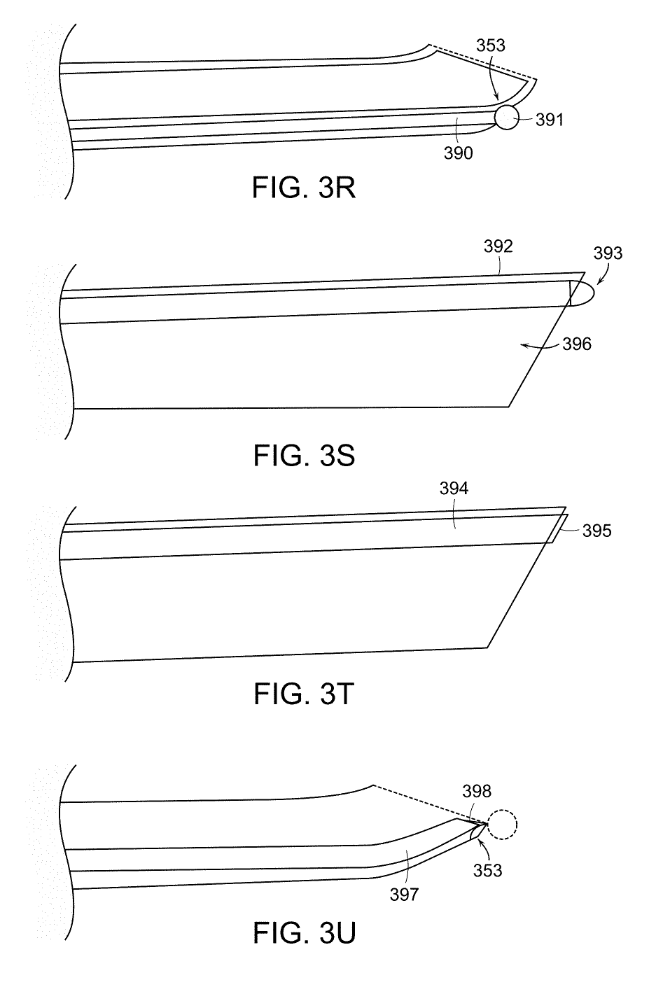

One strategy for probe miniaturization is reducing the wasted area so that the packing ratio can be increased. In various embodiments, multi-layer filter coatings are directly deposited at the tips of fibers in a coating chamber instead of assembling a filter disk and a tube at the tip of the fibers. The direct coating process can be accomplished by modifying the jig to hold fibers instead of micro disks and micro tubes. Since the cost of multilayer coatings is charged per run of the chamber, many fibers can be coated at the same cost. Moreover, the fabrication process will be simplified by removing much of the manual assembly process. This approach miniaturizes the probe, simplifies the fabrication process, and reduces the production cost. A probe shown in FIG. 3A utilizes 40% or more of the probe area for collection and 7% for excitation.

Since sapphire ball lenses are available in various sizes, this direct coating process enables fabrication of a Raman probe with less than 1-mm diameter without significantly compromising its performance. In order to increase the collection efficiency, a transparent sapphire window with 0.75 mm diameter is inserted between the fiber tip and the sapphire ball lens. The optimal thickness is calculated based on computer simulation (ZEMAX).

Several embodiments provide prototype clinical devices that can be used in a clinical setting without, or with minimal, modification of the current epidural procedure. Although a spectroscopy sensor may provide superior performance to the current loss-of-resistance (LOR) method, this new technology will be much more easily adopted by simultaneous use of both the spectroscopic and traditional LOR techniques with minimum modification of the current workflow.

With respect to FIGS. 3A-3Q, various embodiments of needle probes are illustrated. FIG. 3Q shows a side view of a needle used in various embodiments described herein in which three sample detection zones can be accessed. A detection zone is the area that is illuminated and interrogated by the fiber optic probe and may be determined by the choice of associated optical elements. The location of a detection zone may be determined, for example, by the location of the probe with respect to the needle. A first detection zone 301 is a region that lies just adjacent to and slightly above the tip as seen in side view in FIG. 3Q. A second detection zone 303 is a region that lies just adjacent to and slightly below the tip as seen in side view in FIG. 3Q. A third detection zone 305 is a region that lies fully below the tip as seen in side view in FIG. 3. In some embodiments, a detection zone of a fiber optic probe lies less than 2 mm from the needle tip. In preferred embodiments, the detection zone of the fiber optic probe lies less than 1.5 mm from the needle tip and more preferably 1 mm or less from the needle tip. It is preferred that the detection zone at least partially extends to the cutting edge of the device so as to illuminate tissue as it impacts the cutting edge.

FIG. 3A shows a side perspective view of a probe within a needle according to various embodiments. The probe consists of a fiber optic device 54 that sits within a needle 50. The diameter of the fiber optic device 54 is small enough that the lumen 52 of the needle 50 allows fluid flow. Specifically, the needle 50 of the present embodiment can still perform the loss-of-resistance needle placement technique. The fiber optic device 54 comprises a delivery fiber 58 and one or more collection fibers 60. The curved shape 56 of the needle 50 is advantageous to prevent tissue coring and clogging of the needle. However, it is difficult to collect light from the side-facing needle opening with a forward-facing light collection probe 60. To alleviate this problem, the fiber optic device 54 may bend to match the curved shape 56 of the needle near the fiber device's distal end. In addition, the needle may have a viewport 62 near the tip to create another ingress for light to enter the needle. In accordance with various embodiments, the viewport 62 may contain a window made of glass, sapphire, plastic, or another suitable material or it may be a substantially open passage. The viewport 62 allows the collection of light from a forward direction near the tip of the needle 50 while protecting the lumen 52 and the fiber optic device 54 from tissue coring and clogging. To facilitate light collection from and delivery to the tissue, a beamshaping element such as a symmetric or asymmetric reflective mirror or lens 64 may be placed between the fiber optic device 54 and the viewport 62.

FIG. 3B shows an end view of the probe shown in FIG. 3A. According to various embodiments, the viewport 62 may take on any shape, for example but not limited to, that of a circle, square, oval, trapezoid, polygon, or slit. As described above with reference to FIG. 3A, the lumen 52 of the needle 50 remains open to allow for fluid flow through the needle.

FIGS. 3G and 3H show side and end views of a probe within a needle according to various embodiments. In some embodiments, a stylette 320 may be used to fill the lumen 352 of the needle 350 to prevent tissue cores and foreign matter from entering the lumen 352 during insertion. The fiber optic device 354 may be integrated into the stylette 320 or may pass through a hole in the stylette 320. The stylette 320 can surround the probe and otherwise substantially fills the needle lumen 352 and thereby provide longitudinal strength and stiffness to the fiber optic probe 354 to prevent the probe from being forced back into the needle 350 upon insertion into tissue. A metal sheath may be used to enclose the fiber optic probe 354 to prevent buckling or movement of the probe under strain from tissue insertion. In embodiments where the fiber optic probe 354 and stylette 320 are not integral, the stylette 320 may be retracted alone to leave the fiber optic probe 354 in place near the distal tip of the needle 350. The stylette 320 may be removed at any time during a medical procedure to clear the lumen 352 of the needle 350 and allow for the use of the loss-of-resistance (LOR) technique and for fluid transmission. A distal end of the fiber optic probe 354 may be coupled to an optical element 322. The optical element 322 may be made of any transparent but rigid material including, but not limited to, sapphire. A tapered top portion of the optical element 322 can include reflective material to contain the light within the optical element 322 such that light can only enter and exit the fiber within a small region adjacent to the needle tip. As shown in FIG. 3G, the optical element 322 may be positioned just above the bevel tip of the needle (as seen from the side) to maximize detection of tissues immediately adjacent to the needle tip. In a related embodiment, the distal edge of the optical element 322 can include a sharpened or beveled tip to aid in cutting tissue during insertion of the device.

A fiber optic device contained within a needle and having a reflective beamshaping element 324 is shown in FIG. 3L. In accordance with various embodiments, the reflective beamshaping element 324 can be a mirror attached to a stiff, transparent material. The beamshaping element 324 may extend past the tip of the needle 350 and can focus light exiting or entering the fiber optic probe 354. The transparent material may be chosen to have low noise and excellent transparency properties in the spectral range of Raman, UV, infrared, or visible light or another spectral range of interest. For example, the transparent material can be sapphire in various embodiments. In some embodiments, the beamshaping element 324 or a metal housing containing the beamshaping element may include a coupling device such as an attachment lip 325 that hooks over the tip of the needle 350 to provide stability to the beamshaping element 324 and fiber optic probe 354 to prevent them from being forced back into the lumen 352 of the needle 350 during an insertion operation. The attachment lip 325 may be made of the transparent material or metal, for example. The mirror of the beamshaping element 324 can be shaped such that the two conjugate foci of the mirror are located at the needle tip and the collection fiber of the fiber optic probe, respectively. In some embodiments, the mirror can be an ellipsoidal mirror. The tip of the mirror and/or transparent material or the metal tube of the probe may be sharpened or beveled to provide a cutting surface in accordance with various embodiments.

In FIGS. 3M-P, an alternative placement of the fiber optic device 354 is shown wherein the fiber optic device is located below the bevel tip of the needle (as seen in a side view). In these embodiments, a viewport 353 is needed to allow the fiber optic probe 354 optical access to the tissue region adjacent to the needle tip. In some embodiments such as those depicted in FIGS. 3M and 3O, the viewport 353 may be a “backeye” style port typically used to optimally position a cannula or catheter extending from the distal end of the needle 350. Such backeye openings are featured, for example but not limited to, in the EpiSpin II Safety series needles from Pajunk GmbH (Geisingen, Germany) or the ESPOCAN® series Tuohy needles from B. Braun Medical (Bethlehem, Pa.). The backeye can be sealed with a window to allow light to enter and exit through the backeye while preventing the intrusion of tissue into the needle's lumen 352. Many currently available needles have backeye openings that can be several millimeters down the barrel from the needle tip. As such, there are applications in which the fiber optic probe 354 is not always close enough to the tip to optically interrogate tissue properties at the cutting point. To illustrate the danger of tissue sensing measurement errors, note that the depth of the epidural space can be as small as 0.5-1 mm. If the probe is located too far from the cutting point, the cut may proceed all the way through the epidural space before the epidural space can be sensed by the fiber optic probe 354. To mitigate this factor, an optical beamshaping element 326, 328 can be located at the distal end of the fiber optic probe to re-direct the illumination and reception paths to fall closer to the needle tip to preferentially measure tissue as it impacts the cutting edge. In accordance with various embodiments, the optical beamshaping element 328 may contain a beveled edge. In some embodiments, the optical beamshaping element 326 may include a ball lens or other curved optical elements. The ball lens may partially protrude from the backeye such that the lens itself is seated within and seals the backeye to prevent the intrusion of tissue material into the needle's lumen. A stylette 320 can also be used with embodiments containing a backeye. In addition to preventing tissue coring, the stylette 320 can provide stiffness and support to the fiber optic probe to help the probe withstand longitudinal and torsional forces that occur upon insertion of the probe.

As described above, the large distance between the needle tip and the backeye can be addressed by using beamshaping elements 326, 328. Another solution to the problem is to create a custom viewport 353 located proximal to the bevel tip. FIGS. 3N and 3P illustrate side views of probes and stylettes inserted into needles according to various embodiments. The viewports 353 of these embodiments may be located immediately below the bevel tip (as seen from the side) and may be covered by a window to allow light to pass through but prevent the intrusion of tissue into the lumen 352 of the needle 350. The fiber optic probe 354 of these embodiments may include straight fibers, and the location of the probe allows direct detection of tissue properties immediately adjacent to the cutting point. As described above with reference to FIGS. 3M and 3O, optical beamshaping elements 326, 328 may also be used in these embodiments, and a stylette 320 may be used in these embodiments to prevent intrusion of material through the needle's main opening and to provide stiffness and support to the fiber optic probe. In accordance with various embodiments, the optical beamshaping element 326, 328 may include a ball lens or a tapered or beveled feature that directs light between the fiber optic probe and the needle tip.

With reference to FIGS. 3C and 3D, a probe guidance or mounting system may be employed to secure the probe against the wall of the needle and to prevent unwanted twisting or rotation of the probe. FIG. 3C is a cross-sectional view of the embodiment of a probe within the needle as shown in FIG. 3A. The probe may contain one or more collection fibers or fiber bundles 380 and a single delivery fiber or delivery bundle 384. At one or more points on the outer surface of the probe, notches 370 may be created that are shaped to fit with protrusions 372 on the interior wall of the needle 50. The notches 370 and protrusions 372 may run along substantially the entire length of the probe and needle or may be limited to short sections. According to various embodiments, the notches 370 and protrusions 372 may be connected through the use of adhesive, a “snap-fit”, a friction fit, or a shaped fit wherein the cross-section of the notch 370 is shaped, for example, like a dovetail and the protruding track 372 is shaped complementarily. FIG. 3E shows a similar design as FIG. 3C but with a change in the relative positions of the collection fiber 380 and the delivery fiber 384. In light of the present disclosure, it will be apparent to one skilled in the art that one or more collection fibers 380 and delivery fibers 384 may be arranged in any relative orientation that meets application-specific requirements.

FIG. 3D is a cross-sectional view of an alternate embodiment of a fiber optic probe having two collection fibers 380, 382 and a single delivery fiber 384. In accordance with various embodiments, the two collection fibers 380, 382 may both be used for Raman spectroscopy or one fiber may be used for Raman spectroscopy while the other is used for an alternative methodology such as, for example, diffuse reflectance spectroscopy or intrinsic fluorescence spectroscopy. An alternate embodiment is depicted in FIG. 3F wherein the delivery fiber 384 is located substantially between the two collection fibers 380, 382. In light of the present specification, it will be apparent to one skilled in the art that the delivery fiber 384 may be placed in any relationship with respect to one or more collection fibers 380, 382 as needed to meet application-specific requirements.

In some embodiments, the probe can include a single fiber that illuminates the tissue region of interest of a sample and collects light from the sample. The single fiber can enable a simplified probe structure and an increased fill factor of up to 100%. In multi-fiber embodiments, the probe fabrication process can include several steps to assemble a completed probe including mounting the fibers. The probe including the single fiber can eliminate the need for a multi-layer filter coating process at the distal end when one or more sapphire fibers are used. In this case filters can be provided at the proximal end. In addition, the single fiber probe can have a reduced diameter with respect to a multi-fiber probe. In some embodiments, the diameter of the single fiber probe can be less than 1 mm, less than 500 microns, less than 300 microns, or less than 100 microns.

In some embodiments, the single fiber can be made of sapphire. Sapphire produces minimal Raman background signal and is used in some embodiments as the material for lenses and other optical elements. In particular, the Raman emission band for sapphire is predominantly in the low wavenumber region, and Raman emission from sapphire in the typical region for biological Raman fingerprints is minimal. In some embodiments, the single sapphire fiber does not require the application of distal optical filters. Such a configuration is generally not possible using glass fibers.

As shown in FIG. 3R, a fiber optic device including a single fiber 390 can be located below the bevel tip of the needle (as seen in side view). In some embodiments, the port or distal opening 353 may be a “backeye” style port typically used to optimally position a cannula or catheter extending from the distal end of the needle. As described above with reference to FIG. 3M, an optical beamshaping element such as lens or reflector can be located at the distal end of the fiber to re-direct the illumination and reception paths to fall closer to the needle tip to preferentially measure tissue as it impacts the cutting edge.

FIGS. 3S and 3T illustrate probes including a single fiber placed in Crawford epidural needle 392. These can have a fluid delivery channel 396. In some embodiments and as shown in FIG. 3S, an optical beamshaping element such as a full or half-ball lens 393, or another selected portion fabricated by grinding and polishing of the lens to form a flat face, can be placed at the distal end of the fiber to preferentially direct light into and out of the fiber. In the embodiment shown in FIG. 3T, the single fiber 394 is beveled at the tip 395. As light exits the tip of the single fiber, the light will preferentially be directed towards the cutting edge of the Crawford needle.

FIG. 3U shows a probe including a single fiber according to various embodiments. In some embodiments, the single fiber 397 can be beveled at the distal end, and the bevel can be coated with a reflective material to create a mirror-like finish. As illumination light attempts to exit the fiber, it will reflect off of the distal reflecting surface 398 at an angle directed toward the inner wall of the fiber. As this portion of the fiber has no cladding, the Raman illumination light will transmit through the distal opening in the needle wall. Similarly, light entering the fiber at the side wall can reflect light from the bevel that is coupled into the fiber. In these embodiments, an area at the distal end of the fiber can be stripped of all cladding or other coatings to allow light to pass through the side wall of the optical fiber. As shown, the opening 353 of this embodiment may be located immediately below the bevel tip (as seen from the side) and may be covered by a window to allow light to pass through but prevent the intrusion of tissue into the lumen of the needle.

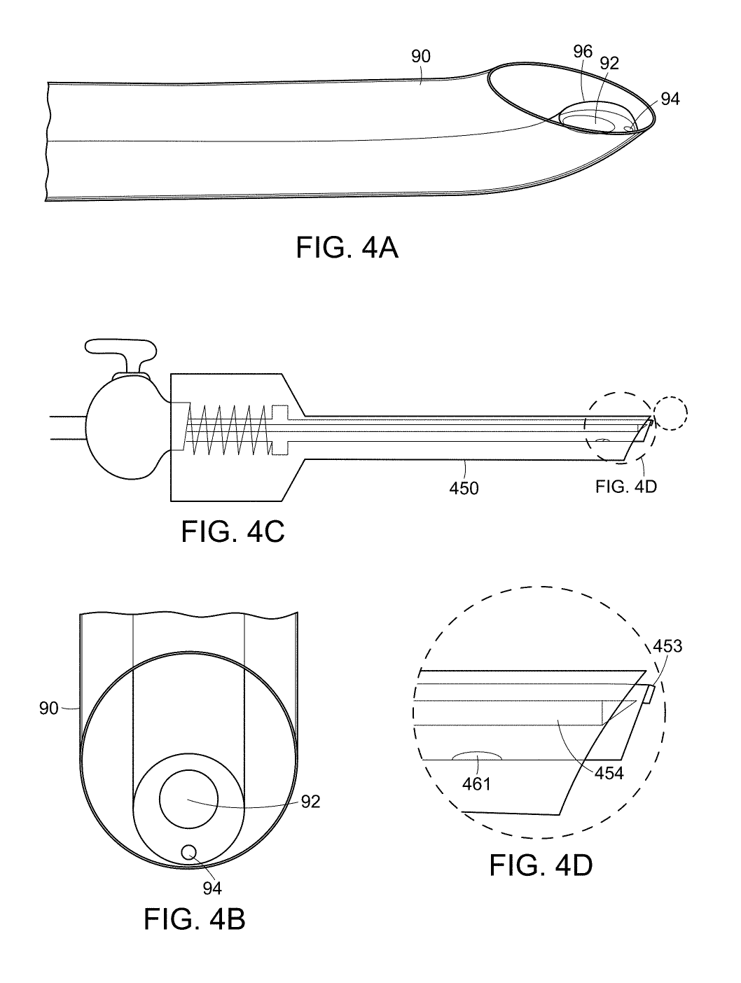

With reference to FIG. 4A, a fiber optic probe may be disposed within a needle 90 such that the distal end of the probe can directly sample the area at the tip of the needle 90. The fiber optic probe comprises a delivery fiber 94 and one or more collection fibers 92. In various embodiments, the distal end of the fiber optic probe can comprise a beamshaping element 96. The beamshaping element may comprise a ball lens, half-ball lens, or other suitable refractive or reflective element as required by the application. FIG. 4B illustrates an end view of the probe with a needle shown in FIG. 4A. According to various embodiments, the collection fiber 92 may be much larger in diameter than the delivery fiber 94. In accordance with certain embodiments, the lumen of the needle is not obstructed by the probe such that fluid can still flow through the needle lumen.

An epidural placement procedure may be performed as follows. A needle can have a stylette 320 and a fiber optic probe 354 placed within as in the embodiments of FIGS. 3G and 3H as described above, for example. Although the present discussion focuses on the embodiment of 3G, those skilled in the art will appreciate that any of the needle embodiments described herein can be used in the procedure described. The needle 350 is advanced into the body of a patient while multimodal spectroscopy measurements are acquired and analyzed to determine the position of the needle tip. At any time, the stylette 320 may be removed (FIG. 3I) to allow supplementary use of the loss-of-resistance technique to determine needle tip placement. When the needle tip is determined to be located in the epidural space, needle advance is halted and the fiber optic probe 354 and/or stylette 320 (if not removed in a prior step) are removed from the needle lumen 352 (FIG. 3J). A catheter 330 may be advanced into the needle lumen 352 to administer therapeutic agents to the epidural space through a catheter lumen 332 as shown in FIG. 3K. The catheter can be flexible and have a single lumen, multiple lumens, a single distal opening, or multiple openings along the catheter sidewall as necessary to meet application-specific requirements. In some embodiments, the catheter may include a wire-steering mechanism.

FIGS. 4C-4D show a cross-sectional side view and a magnified view, respectively, of a device containing a probe placed within a Veress-type trocar assembly for laparoscopy, according to various embodiments. The probe may extend through a spring-loaded blunt stylette 460 that can be placed inside a needle or cannula 450. The spring-loaded blunt stylette 460 may have an outlet 461 at the distal end that allows passage of an insufflation gas such as CO₂ that may be used to inflate the cavity for a surgical procedure and endoscopic visualization. Alternatively, the outlet 461 may allow other medicaments to pass into the abdominal cavity. The device may also be equipped with a gas or liquid valve 462 to control the flow of CO₂ or other medicaments through the catheter. As the needle or cannula 450 is advanced into the body, the spring-loaded blunt stylette 460 is pushed into the needle or cannula but maintains contact with the tissue surface due to the force of the spring. When the tip of the needle or cannula 450 penetrates the peritoneum, the spring force is fully released and the blunt stylette 460 advances forward and out of the cannula 450.

In several embodiments, further miniaturized probes with a 0.5 mm diameter can be employed. These probes can be inserted in needles as small as 21-gauge (0.54-0.58 mm inner diameter). When used with a 17-gauge Tuohy epidural needle, 81-82% of the inner needle space will remain available for simultaneous LOR. The increase in pressure caused by reduced area in the needle can be adjusted by an adapter at the tip of the LOR syringe such that the clinician will feel exactly the same pressure with or without the sensor inside.

Probe dimensions can be reduced by replacing existing components with smaller ones. For example, 200 μm diameter fibers (both excitation and collection) can be replaced by 100 μm diameter fibers with the same numerical apertures. Sapphire ball lenses may be replaced with smaller lenses. The thickness of the sapphire plate can be recalculated to maximize the collection efficiency with reduced form factors. In principle, the probe can be further miniaturized since multimode fibers with the same numerical apertures are available down to a 50 μm diameter. Further, the reduced collection area collects less Raman scattered light from the tissue.

The complicated design of the Raman probe mostly originates from fluorescence background signal generated from the optical fibers. The purpose of the two types of filters at the tip of the fiber probe is to minimize the unwanted background signal; this signal is unavoidable as long as silica fibers are used. Sapphire fibers and hollow-core photonic crystal fibers can be used in the probe of the present invention to eliminate the use of a collection filter at the distal end.

Sapphire has very low background signal and is being used as the ball lens, beamshaping element or window in several embodiments for this reason. However, drawing an optical fiber made out of sapphire has been a challenge for the industry. Recently, a few companies (e.g., MacroMaterials, Inc.) have begun to produce single crystal sapphire optical fibers (75 μm-500 μm diameters) with similar numerical aperture numbers as standard multimode fibers. Ideally, there is no need to separate the delivery and collection fibers. Gluing a sapphire ball lens at the tip of the sapphire fiber can be comparable to other embodiments using multiple fibers. Moreover, 100% of the probe area can be used for collection. Therefore, higher collection efficiency can be achieved.

A second option is a hollow-core photonic crystal fiber. The delivery fiber generates much stronger background signal than the collection fibers due to the higher photon density in the fiber. A Raman probe for needle guidance can utilize hollow-core photonic crystal fibers. The excitation beam is delivered through the air gap in the center of the hollow core fiber and does not generate a background signal as the background is mainly generated from the interaction between laser light and silica.

The Raman probe for using a clinical Raman system delivers light from a 830 nm diode laser that is launched into the excitation fiber of the probe. After filtering the fiber background using a laser line filter at the fiber tip, the excitation beam is focused onto the sample by a sapphire ball lens. Backscattered Raman signal (Rayleigh signal is filtered by a long pass filter) is collected by the six collection fibers connected to an imaging spectrograph. The proximal linear array of collection fibers from the Raman probe can be connected to a f/1.8i imaging spectrograph, which collimates the light before it is notch filtered (NF), focused onto a slit, and recollimated for dispersion by the holographic grating (HG). Finally, the dispersed light can be focused onto a TE-cooled, back-illuminated, deep depletion CCD detector, which is interfaced with a laptop computer.

Probes can be too large to fit through many procedural needles used clinically. Therefore, a preferred system embodiment provides an ultra-thin optical probe with improved optical throughput and efficient chemometric algorithms to compensate for the signal loss in a small device. In some embodiments, the probe is miniaturized to a diameter of less than 1 mm. A near infra-red diode laser can be used as an excitation source. The standard epidural needle is modified to hold an RS probe. The collected signal is delivered to a compact Raman spectrometer. A tablet or laptop computer controls the hardware and analyzes the data for real-time feedback. The epidural needle with an RS probe is inserted along the two traditional epidural needle placement trajectories (midline and paramedian approaches). The device aids in differentiating tissues during tissue needle biopsies, peripheral nerve blockade, lumbar puncture, laparoscopic trocar placement, orthopedic joint injection, and drainage of fluid collections in the abdomen and thorax.

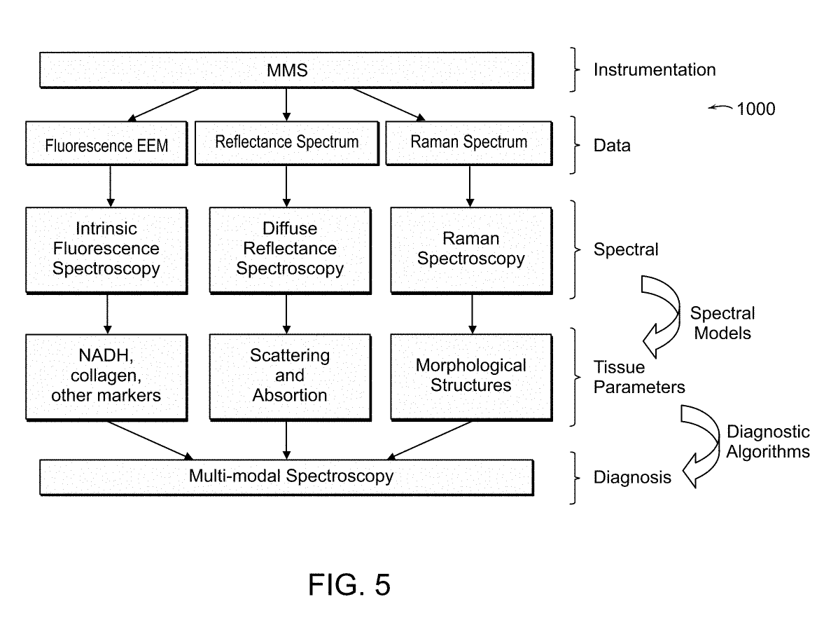

An MMS system 1000 is generally illustrated in FIG. 5. The MMS system 100 comprises multiple modalities that come together to provide a single diagnostic evaluation. Data is acquired from the tissue source in the form of filtered light. The optical filter regime is chosen according to the modality that is desired: Raman spectra, fluorescence excitation-emission, and diffuse reflectance. This raw data is processed to obtain usable spectra that may be compared to standards for identification. By comparing several key markers in each of the processed spectra to basis sets, tissue parameters can be extracted. For example, a Raman spectrum can determine the morphological structure of the tissue under study while DRS and IFS can contribute information about scattering and absorption and specific biomarkers in the system, respectively. By applying diagnostic algorithms to the spectra and the tissue parameter findings, a diagnosis is made.

In order to provide real-time spectroscopic based diagnosis, spectra for calibration and background subtraction are acquired prior to data collection. The Raman shift axis is calibrated by collecting a spectrum from a known Raman scatterer, 4-acetamidophenol (Tylenol). The Raman fiber background signal is measured through acquisition of a spectrum from a roughened aluminum block. The spectral response of the Raman collection system is obtained by recording the spectrum of a calibrated tungsten white light source diffusely scattered by a reflectance standard (BaSO4).

The system throughput may be quantified by measuring homogenous liquid samples. Liquid tissue simulating phantoms will be prepared and measured. Reducing probe size generally results in reduced throughput. However, comparable throughput can be achieved due to the increased packing ratio.

The sampling volume can be characterized by measuring a bilayer solid tissue simulation phantom. Two-layer gelatin tissue phantoms are prepared with the same optical properties. Scattering and absorption parameters may be controlled by an intralipid solution and trypan blue, respectively. To generate Raman contrast between the two layers, glucose may be added to one layer. While the probe is progressing from the first to the second layer by micrometers, Raman spectra are acquired. The Raman signal change is analyzed as a function of movement, and axial resolution is calculated. One advantage of a smaller fiber probe is reduced sampling volume (better axial resolution). Improved axial resolution provides better contrast between each tissue layer. The effects of the design parameters (probe cross section and packing ratio) can be calculated by using a Monte-Carlo simulation. Measurement results can then be compared to the simulation values to ensure high fabrication quality.

Tissue for measurements was obtained from 40-50 kilogram, male and female, 3-4 month old Yorkshire swine. Immediately following euthanization, the spinal column corresponding to the lumbar and lower thoracic regions was excised en bloc and refrigerated at 4° C. until measurements and final dissection. All tissue was refrigerated until use, used within 24 hours of euthanization, and scanned at room temperature. The custom-built 1 mm probe within a 17-gauge Tuohy epidural needle was inserted in the spinal column at the midline between spinous processes and lateral to the midline in order to mimic the two commonly used approaches to epidural space localization with the traditional loss of resistance (LOR) technique. Raman spectroscopy (RS) data was recorded continuously (data acquisition approximately once per second) during epidural needle insertion. The probe-in-needle was advanced at 1 mm increments starting at the skin, through the epidural space, and ending at the spinal cord in order to acquire data from all tissue types along each of the two trajectories. One millimeter increments were used in order to accurately identify each transition from one tissue type to another as some of the tissue layers are approximately 1 mm thick. Thus, incremental advances in a range of 0.5-2 mm are used to provide an effective sampling rate.

The initial probe-in-needle insertion measurements used radiographic (x-ray) guidance in order to accurately position the needle in 1 mm increments from the skin to the spinal cord. In addition, the radiographic images further confirmed the tissue type that the needle was in and corroborated the spectroscopic data. Subsequent probe-in-needle insertions did not use x-ray guidance in order to decrease operator bias. Finally, a number of needle insertions were performed using optical spectra interpretation alone in order to assess the ability to localize the epidural space by this technique alone.

Initially, Raman spectra from various ex vivo tissue samples were acquired using a custom-built NIR Raman microscope. Briefly, a 785-nm wavelength Ti:Sapphire laser was used as an excitation source and XY tissue scanning was performed by the galvanometer mirrors. A 1.2 NA water immersion objective lens was used to focus the laser light onto the sample and to collect the back-scattered light. The back-scattered Raman light from the sample passes through two dichroic mirrors and was collected by a multi-mode fiber and delivered to the spectrograph and detected by a thermoelectric-cooled, back-illuminated and deep depleted charge-coupled device (CCD). LabView 8.6 software (National Instruments), data acquisition board (PCI-6251, National Instruments), and MATLAB 2013 software (Mathworks) were used to control the system, acquire the data, and analyze the data. Dissected tissues were placed on top of quartz coverslips (043210-KJ, Alfa Aesar). Twenty-five (5×5) spectra were acquired from a 38 μm×38 μm area in order to confirm tissue inhomogeneities. Sixty milliwatts of power was delivered to the sample with a 5 sec integration time.

Diffuse reflectance spectra were acquired by a tissue scanner (see FIG. 20). A broad-band laser driven light source (EQ-99-FC-S LDLS, Energetiq Technology) was used as an excitation source. XY scanning was performed by a motorized stage (MS-6000-XY, Applied Scientific Instrumentation). An optical fiber probe (one central excitation fiber and six surrounding collection fibers) was used to both deliver the broad band light onto the sample and to collect the back-scattered light. The back-scattered reflectance light from the sample was delivered to a miniature spectrometer (HR-2000+, Ocean Optics). LabView 8.6 software (National Instruments) and MATLAB 2013 software (Mathworks) were used to control the system and analyze the data. Dissected tissues were placed on top of a quartz plate. From each tissue, 25 (5×5) spectra were acquired from a 2.5 mm×2.5 mm area in order to confirm tissue inhomogeneities. Fifteen milliwatts of power was delivered to the sample with a 1 millisecond integration time.

Fluorescence spectra were acquired by the tissue scanner. A 355-nm UV laser (SNV-40E-000, Teem Photonics) was used as an excitation source. The optical fiber probe (one central excitation fiber and six surrounding collection fibers) was used to both deliver the UV light onto the sample and to collect the back-scattered light. The back-scattered fluorescence light from the sample was delivered to a miniature spectrometer (HR-2000+, Ocean Optics). LabView 8.6 software (National Instruments) and MATLAB 2013 software (Mathworks) were used to control the system and analyze the data. Dissected tissues were placed on top of a glass plate. From each tissue, 3 spectra were acquired from 3 different locations in order to confirm tissue inhomogeneities. Three milliwatts of power was delivered to the sample with a 100 millisecond integration time.

The excised spinal column can be dissected to separate the following tissues: epidermis/dermis, fat, skeletal muscle, supra-/intra-spinous ligament, ligamentum flavum, epidural fat, dura mater, and spinal cord. Each sample is independently scanned with RS. After scanning, each sample is placed in 10 milliliters of formalin, embedded in paraffin, cut, placed on slides, and stained with hematoxyline and eosin. In order to confirm tissue types, all slides are reviewed and identified by a qualified neuropathologist. Finally, data from the probe-in-needle insertions and from the dissected tissues are compared to previously acquired tissue Raman spectroscopy data.

A comparison of the three spectroscopy modalities used to examine neuraxial and overlying tissues is seen in Table 1. Given the limitations of DRS and IFS and the need to differentiate each tissue from the skin to the spinal cord (epidermis/dermis, fat, supra-/intra-spinous ligament, ligamentum flavum, dura mater, spinal cord), Raman spectroscopy provides a preferred modality for differentiation of these tissues. Nonetheless, we measured dissected tissues with all three spectroscopic modalities.

TABLE 1
Comparison of multi-modal spectroscopy methods.

| | DRS | IFS | RS |
|---|---|---|---|
| Measurement | white light reflectance | UV-induced fluorescence | NIR Raman spectrum |
| Tissue | oxy-/deoxy-hemoglobin, | Collagen, | DNA, |
| properties | β-carotene, | elastin, | protein, |
| | melanin, | keratin, | lipid, |
| | scattering parameters | NADH, | +all the chemicals from |
| | (A, B, C) | FAD | DRS and IFS |
| Advantage | Strong signal | Sensitive to connective | Comprehensive chemical |
| | Simple instrumentation | tissues | information |
| Disadvantage | Heavy modeling, | Requires DRS for | Relatively weak signal |
| | sensitive to measurement | analysis, | |
| | geometry | Photobleaching | |

Depending upon the application, DRS spectra will not readily exhibit clearly visible differences among tissue layers. DRS is an interplay between tissue absorption and scattering. Therefore, even small amounts of tissue inhomogeneity and/or small variations of probe-sample distance can affect the analysis result. In certain applications, this may be a critical drawback for reflection-based needle-tip tissue diagnosis because small adjustments in the location of the inserted needle are necessary, and such adjustments can alter results.

IFS can detect fluorescence signals from connective tissues (i.e., skin, supra-/intra-spinous ligament, ligamentum flavum, and dura mater). However, IFS cannot provide information about some other types of tissues (e.g., fat, muscle, and spinal cord) nor can it particularly differentiate the connective tissue layers from one another. An additional drawback to IFS in certain applications is that IFS suffers from photobleaching, which can limit the ability to make quantitative measurements.

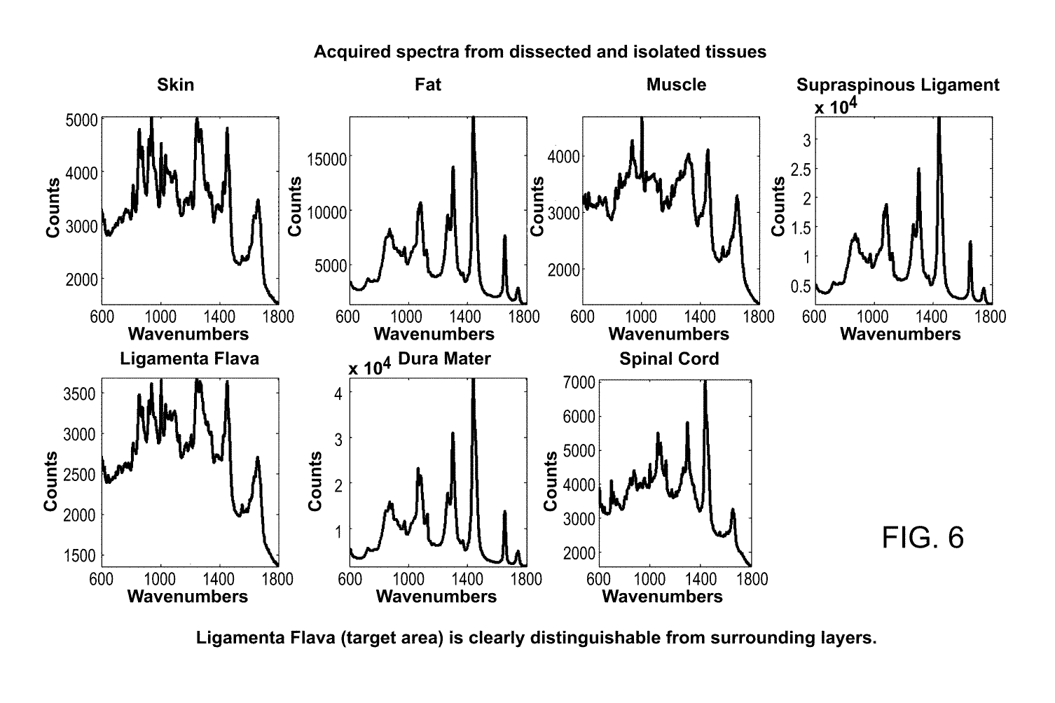

The averaged spectra from dissected tissues are shown in FIG. 6. Raman spectra from eight dissected tissues (epidermis/dermis, fat, skeletal muscle, supra-/intra-spinous ligament, ligamentum flavum, epidural fat, dura mater, and spinal cord) are plotted with the same y-scale. Two highlighted Raman bands represent connective tissues and fatty tissues. The first highlighted Raman band represents 939 cm⁻¹ from collagen. A collagen Raman band is found in the spectra of epidermis/dermis, supra-/intra-spinous ligament, ligamentum flavum, and dura mater. These are the same layers where IFS showed strong collagen fluorescence signals. The second highlighted Raman band represents 1450 cm⁻¹ from lipids. Strong lipid signals were observed in the spectra from fat and epidural fat.

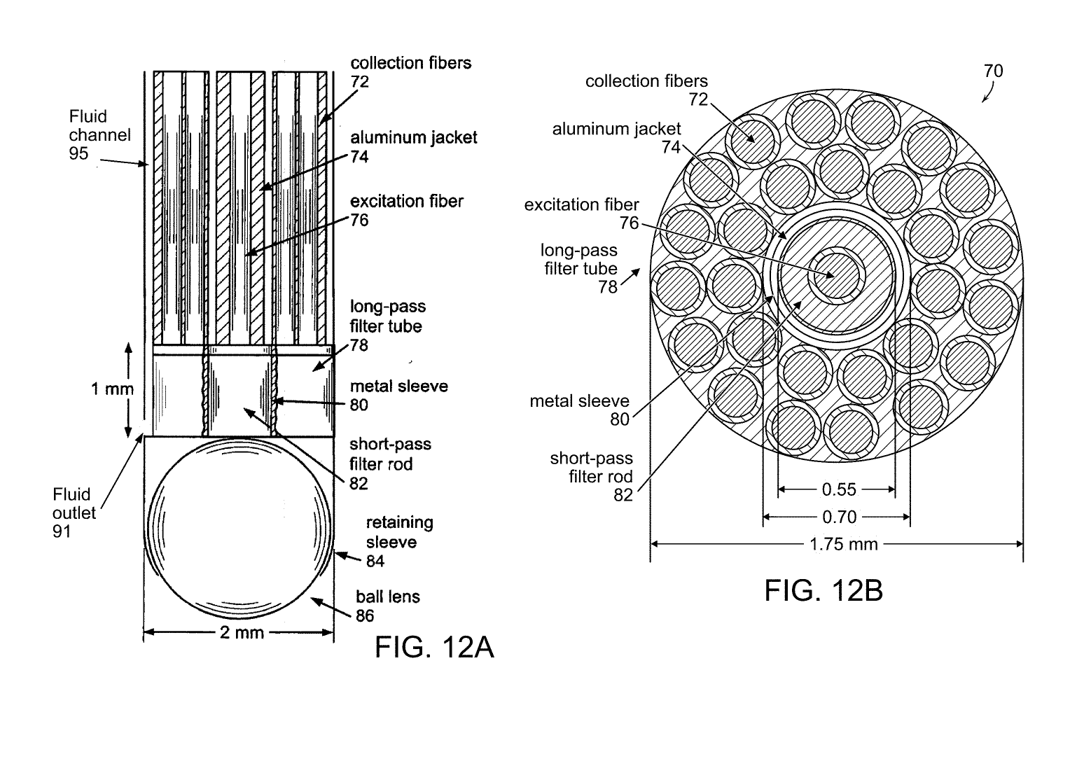

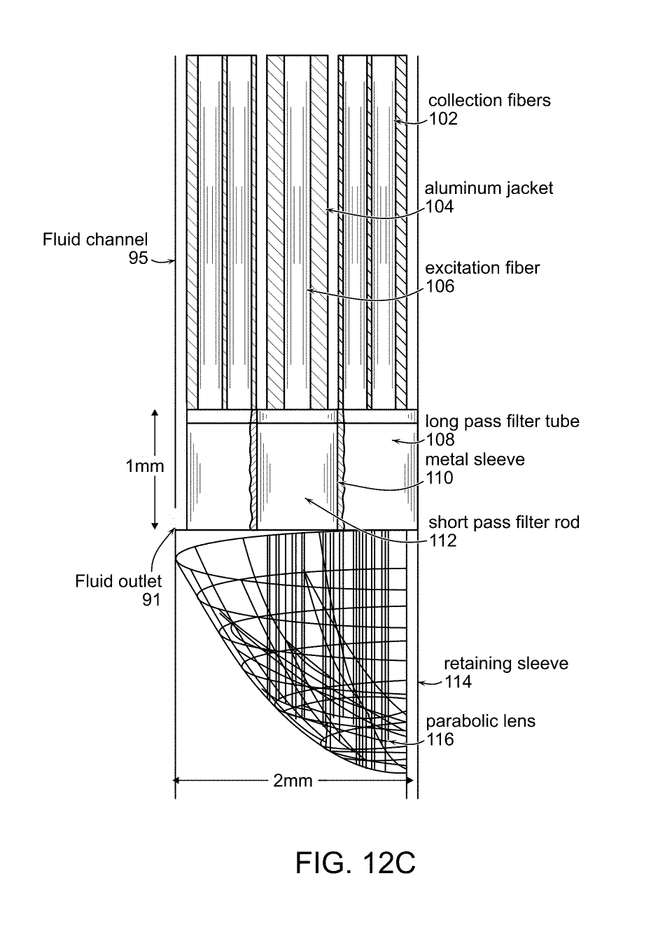

Next, Raman spectra were acquired by a clinical multimodal spectroscopy system according to the present invention. The instrument uses an 830 nm diode laser to excite Raman scattering. The laser light is filtered and coupled to an optical fiber Raman probe which delivers light to, and collects light from, tissue. The Raman probe contains a single excitation fiber surrounded by a ring of 15 collection fibers, specialized filters on both the excitation and collection fibers at the distal tip of the probe, and a ball lens at the tip to optimize signal collection. A two-millimeter-diameter Raman probe was integrated into a two millimeter inner diameter needle (see FIG. 12A-12C) for measurement of epidural needle advancement to the epidural space. The return light is passed through a spectrograph and dispersed onto a charge-coupled device (CCD) detector. As previously described, following swine euthanization, the spinal column corresponding to the lumbar and lower thoracic regions was excised en bloc and refrigerated at 4° C. until use. A needle and stylette were inserted into the tissue and Raman spectra were obtained at 1-2 mm intervals from skin to spinal cord. After each 1-2 mm movement, the stylette was removed, the RS probe was inserted, and RS spectra obtained. The stylette was reinserted prior to the next 1-2 mm step to prevent tissue coring. Ten spectra were acquired from each layer with 100 mW of power and a 1 second integration time. The needle was advanced along trajectories that correspond to the midline and paramedian approaches to epidural space localization.

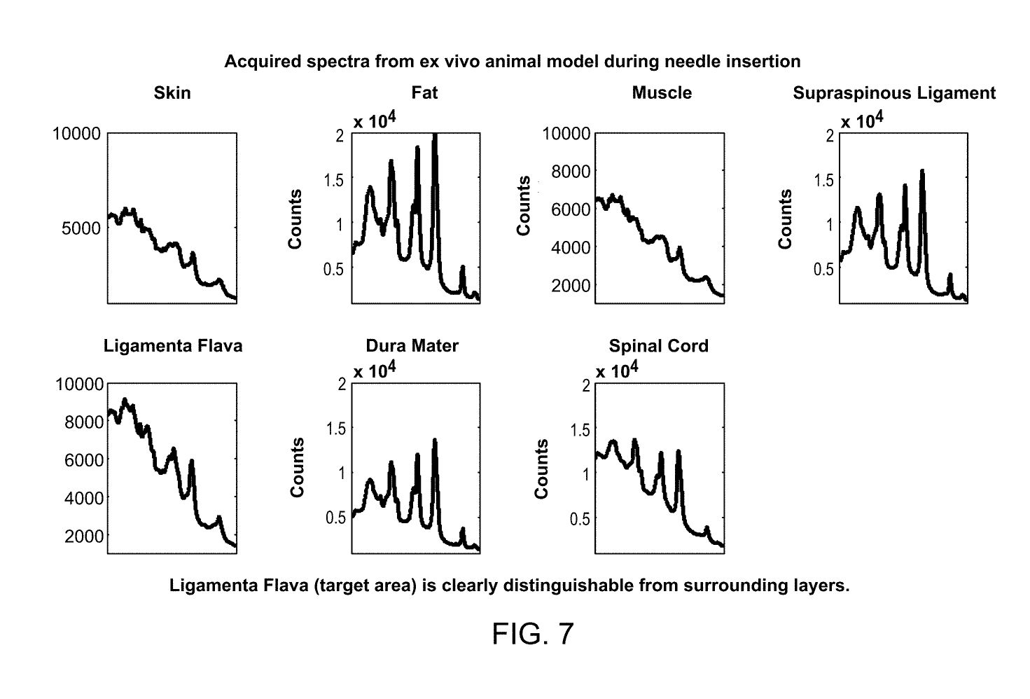

A 2-mm ID needle was advanced along trajectories that correspond to the midline and paramedian approaches to localization of the epidural space with the traditional LOS technique. RS spectra were acquired at intervals and are shown in FIG. 7. RS spectra were obtained from epidermis/dermis, fat, skeletal muscle, supra-spinous/intra-spinous ligament, ligamentum flavum, and epidural fat. Overall the spectra have a smoother profile than those from dissected tissues due to different spectral resolutions (4 cm⁻¹ for dissected tissue measurements vs. 16 cm⁻¹ for needle-tip measurements) of the two instruments. Nonetheless, the RS spectrum of each tissue is unique compared to the other tissues studied and similar to those acquired from the individually dissected tissues.

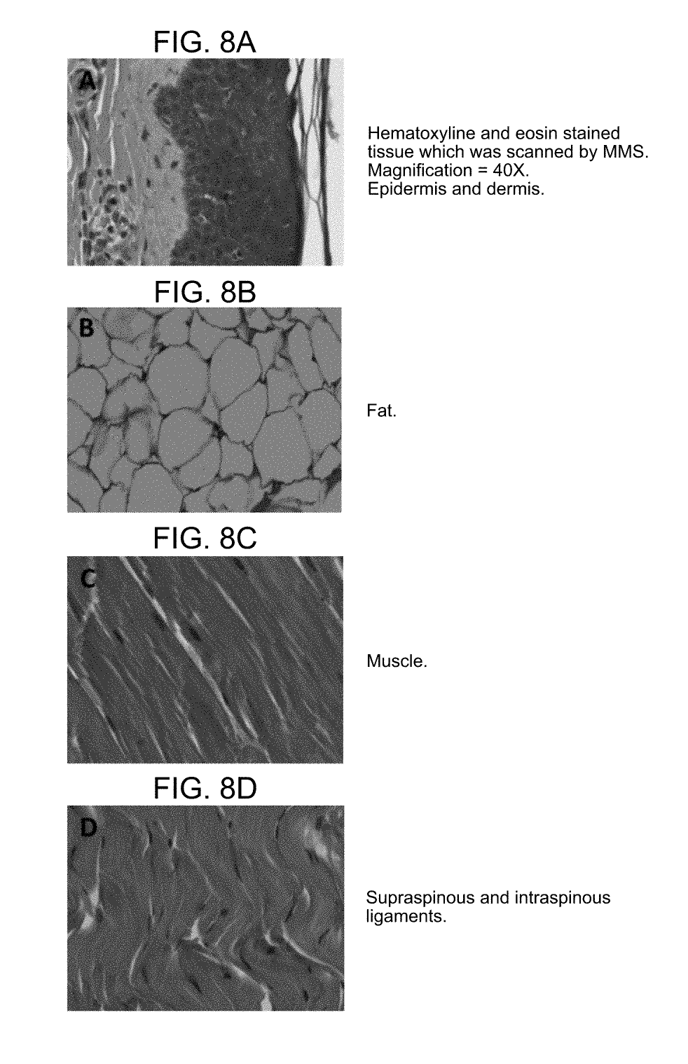

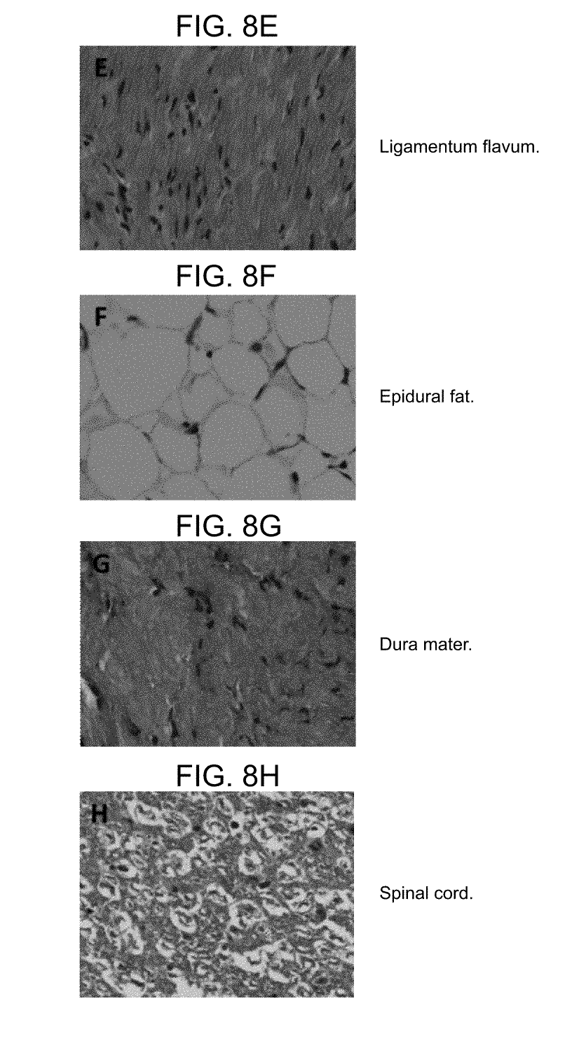

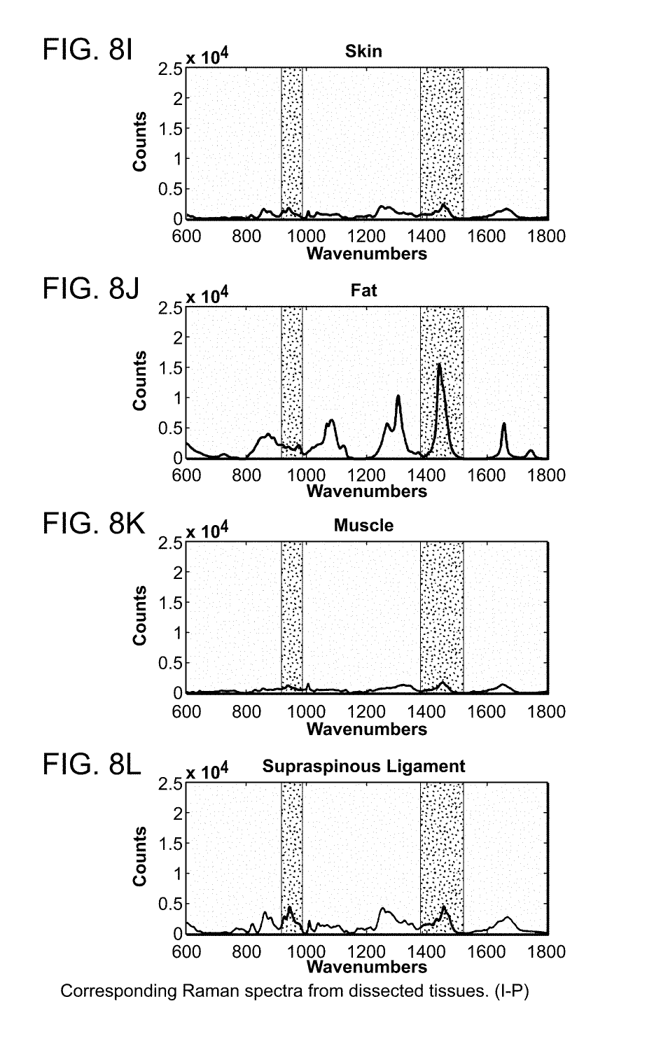

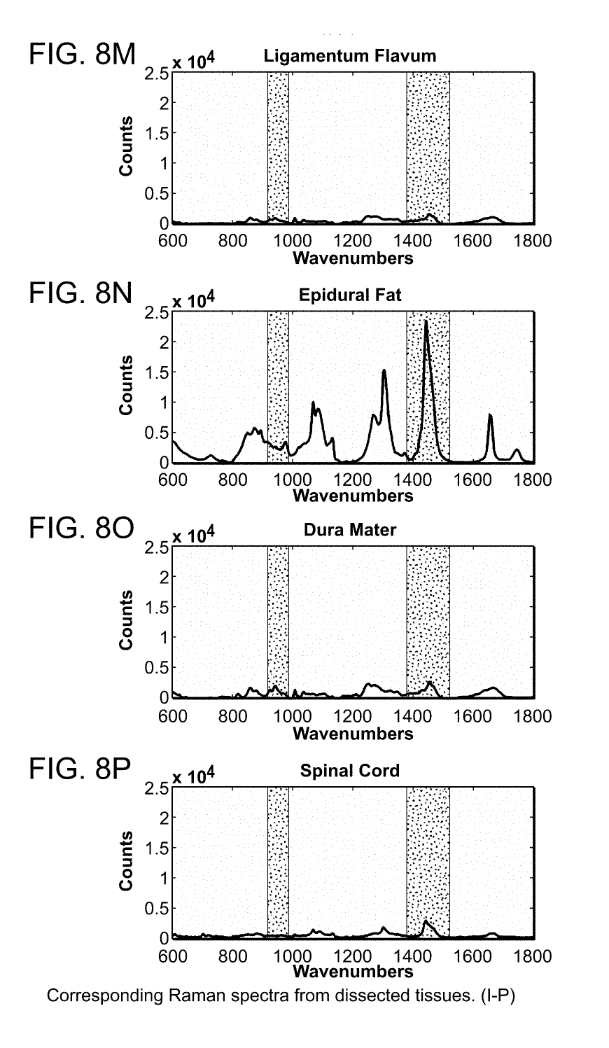

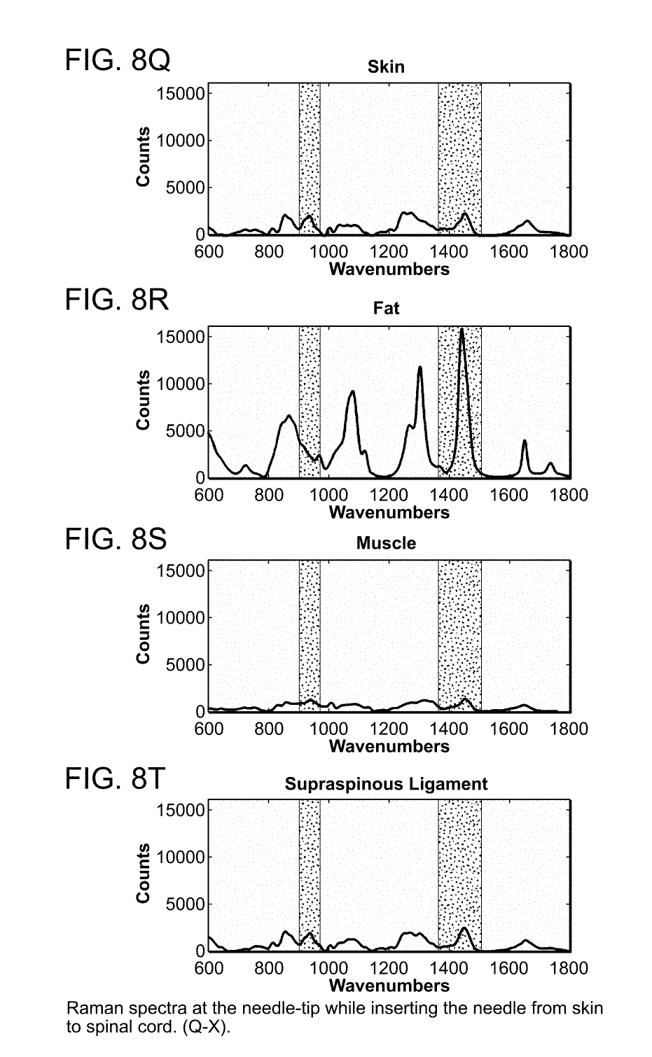

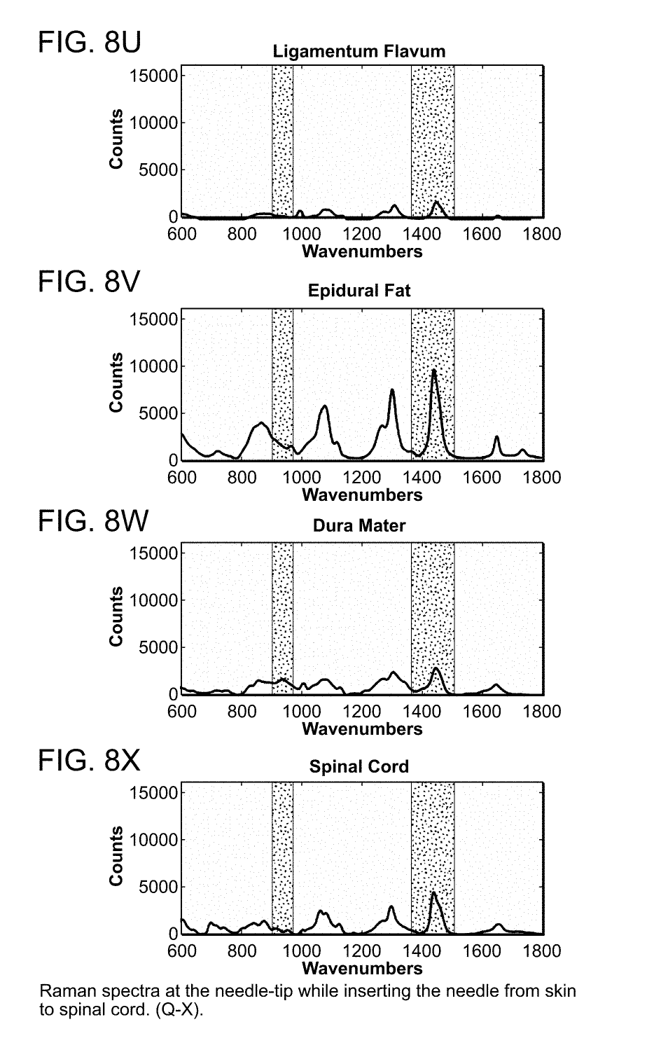

The tissues identified during dissection, and subsequently scanned by Raman microscopy, were confirmed by histology. Epidermis/dermis, fat, skeletal muscle, supraspinous/intraspinous ligament, ligamentum flavum, epidural fat, dura mater, and spinal cord underwent H & E staining and were independently assessed by a neuropathologist who confirmed the tissue type (FIG. 8).

Compared to other optical methods, the advantage of Raman spectroscopy is the huge amount of information gathered during scanning. Each Raman band provides molecular bonding information from the sample. Generally, 10-20 Raman peaks are clearly visible from tissue Raman spectra. Correlating these Raman band intensities with individual tissues is the major task for a decision algorithm. Both hardware and software are critical for successful translation of the technology to the medical field. In certain embodiments, several types of decision algorithms are used for tissue identification during epidural needle placement.

The first algorithm is based on a linear decomposition of acquired spectra into basis spectra. These models were developed and applied for two diseases (atherosclerosis and breast cancer). One of the key requirements for successful morphological modeling is that there needs to be very little inter-sample variation in the Raman spectra of a given morphological structure. The measured Raman spectra of tissue layers along the path of an epidural needle (epidermis/dermis, fat, skeletal muscle, supra-/intraspinous ligament, ligamentum flavum, epidural fat, dura mater, and spinal cord) must all be used to ensure that the model includes the common elements of all morphological features. The key to successful fitting is to use as few elements as possible while retaining relevant spectral information in order to avoid over-determining the spectrum. The fitting coefficients from the basis spectra are used to define each tissue layer.

The second algorithm is based on nonlinear methods. Acquired spectra with histologic identification are used as a training dataset. After completing the training model, leave-one-out cross validation is performed to check the model's prediction capability. Ultimately, this prediction model can be applied to newly acquired data sets (prospective prediction) and is confirmed by gold standard histologic results. The performance of the two independently developed algorithms can be compared, and the better one can be selected for further use in the device.

Finally, decision algorithms based on selected Raman bands have been developed. Generally, more than half of the Raman spectrum does not have visible Raman peaks. Further, Raman peaks from biological samples are relatively broad compared to sharp Raman peaks from pure chemicals. This suggests the possibility of developing a Raman spectroscopy system based on a limited number of discrete Raman bands. With full spectrum information from previous measurements, a discrete Raman band-based tissue identification algorithm can be developed and its performance compared to the full spectrum methods. This new approach can potentially reduce the size and cost of the device by replacing the spectrograph and CCD with optical filters and photodetectors.

For quantitative analysis, acquired Raman spectra from dissected tissues were decomposed as a summation of basis components. Each acquired spectrum was represented by five fitting coefficients that represent relative proportions of basis components. The methods described herein can be used for numerous applications to guide procedures within a patient including a biopsy needle to a tissue exhibiting a pathology requiring tissue sample removal or to guide a nerve block procedure. This can involve nerve blocks in peripheral locations as well as other locations such as the main torso, neck or to prevent pain associated with a patient's respiration such as for the brachial plexus. A local anesthetic can be applied and an optical needle inserted to guide delivery to the proper location, optionally using an ultrasound guided technique in combination.

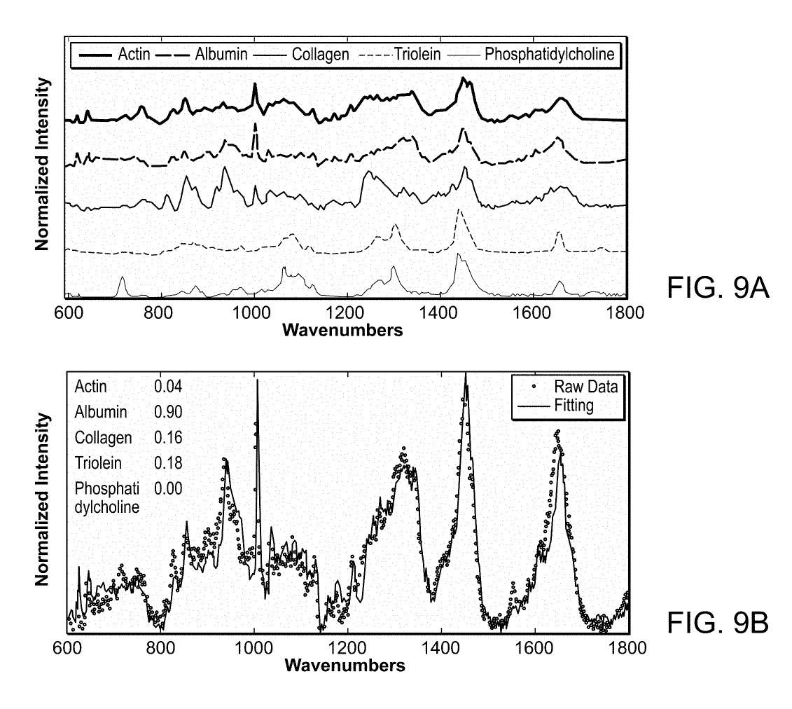

Raman spectra from five chemicals (actin, albumin, collagen, triolein, and phosphatidylcholine) were normalized and selected as basis spectra (FIG. 9A). Each Raman spectrum was represented as a vector. In order to test the orthogonality of the basis spectra, the degree of orthogonality was calculated by the below equation.

$$\frac{{x}^{T} y}{\sqrt{( {x}^{T} x ) ( {y}^{T} y )}}$$

where x and y represent two basis components. Zero means fully orthogonal while one means they are identical. The calculated values are summarized in Table 2. All components have values >0.7 compared to one another. This commonality is due to the common vibration modes (for example, CH₂ or C—C vibrations). Still, the components were well differentiated when using ordinary least-squares fitting.

TABLE 2
Degree of orthogonality of the five basis components.

| | Actin | Albumin | Collagen | Triolein | Phosphatidylcholine |
|---|---|---|---|---|---|
| Actin | 1 | | | | |
| Albumin | 0.95 | 1 | | | |
| Collagen | 0.92 | 0.89 | 1 | | |
| Triolein | 0.84 | 0.78 | 0.77 | 1 | |
| Phosphati- | 0.83 | 0.77 | 0.77 | 0.94 | 1 |
| dylcholine | | | | | |

For biochemical decomposition of the tissue, the ordinary least squares fitting method was used. To avoid data overfitting, the fitting process was carefully monitored by restricting the fitting coefficients to non-negative values. Acquired Raman spectra were normalized and decomposed as a summation of basis components. FIG. 9B demonstrates a fitting result for a spectrum acquired from muscle tissue. The fitting spectrum (red line) is similar to the raw data (blue dots), and the five fitting coefficients provide the chemical composition of measured tissue.

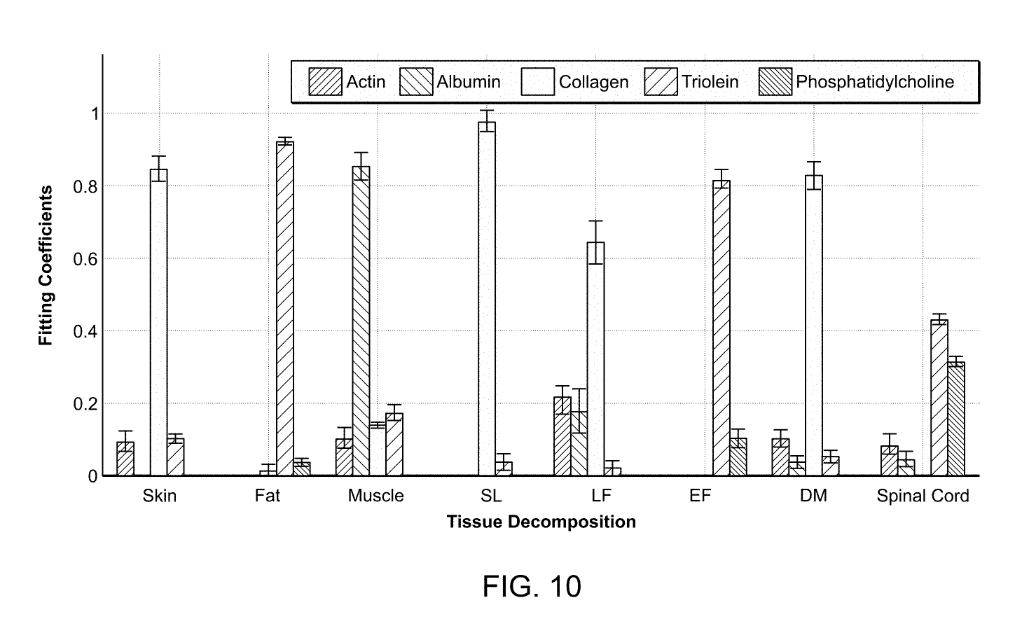

Quantitative analysis was performed on the dissected tissues using ordinary least squares fitting. The acquired tissue spectra were decomposed into five basis spectra (actin, albumin, collagen, triolein, and phosphatidylcholine). Each tissue layer is represented as five fitting coefficients of five basis components. As a result, Raman spectra from dissected tissues resulted in 25 coefficients for each basis spectrum. The fitting coefficients of eight tissue layers are shown in Table 3 and FIG. 10. It should be noted that the fitting coefficient of each basis component stands for a “relative” amount of basis components since each basis component has different Raman cross section. For example, skin has a 0.09 fitting coefficient for actin and 0.84 for collagen. This does not mean there is 9 times more collagen components compared to actin components since collagen and actin have different Raman cross-sections. On the other hand, 0.22 (actin) and 0.64 (collagen) fitting coefficients for the ligamentum flavum tells that there is 144% more actin and 24% less collagen compared to the skin tissue layer.

TABLE 3
Fitting coefficients of eight tissue layers.

| | Actin | Albumin | Collagen | Triolein | Phosphatidylcholine |
|---|---|---|---|---|---|
| Skin | 0.09 ± 0.03 | 0.00 ± 0.00 | 0.84 ± 0.03 | 0.10 ± 0.01 | 0.00 ± 0.00 |
| Fat | 0.00 ± 0.00 | 0.00 ± 0.00 | 0.01 ± 0.02 | 0.92 ± 0.01 | 0.03 ± 0.01 |
| Muscle | 0.10 ± 0.03 | 0.85 ± 0.04 | 0.14 ± 0.01 | 0.17 ± 0.02 | 0.00 ± 0.00 |
| Supraspinous Ligament | 0.00 ± 0.00 | 0.00 ± 0.00 | 0.98 ± 0.03 | 0.04 ± 0.02 | 0.00 ± 0.00 |
| Ligamentum Flavum | 0.22 ± 0.03 | 0.18 ± 0.06 | 0.64 ± 0.06 | 0.02 ± 0.02 | 0.00 ± 0.00 |
| Epidural Fat | 0.00 ± 0.00 | 0.00 ± 0.00 | 0.00 ± 0.00 | 0.82 ± 0.03 | 0.11 ± 0.02 |
| Dura Mater | 0.10 ± 0.03 | 0.04 ± 0.02 | 0.83 ± 0.04 | 0.05 ± 0.02 | 0.00 ± 0.00 |
| Spinal Cord | 0.09 ± 0.03 | 0.05 ± 0.02 | 0.00 ± 0.00 | 0.43 ± 0.01 | 0.32 ± 0.01 |

Using actin, albumin, collagen, triolein, and phosphatidylcholine as pure basis spectra, tissue layers are composed of a mixture of these five components. Epi-/dermis signal is mainly a combination of actin, triolein, and collagen. Ligamentum flavum is a mixture of actin, albumin and collagen. Interestingly, epidural fat can be differentiated from subcutaneous fat from the ratio of triolein and phosphatidylcholine. The spectrum from dura mater is a mixture of the spectra of actin and collagen but includes higher collagen content in comparison to ligamentum flavum.

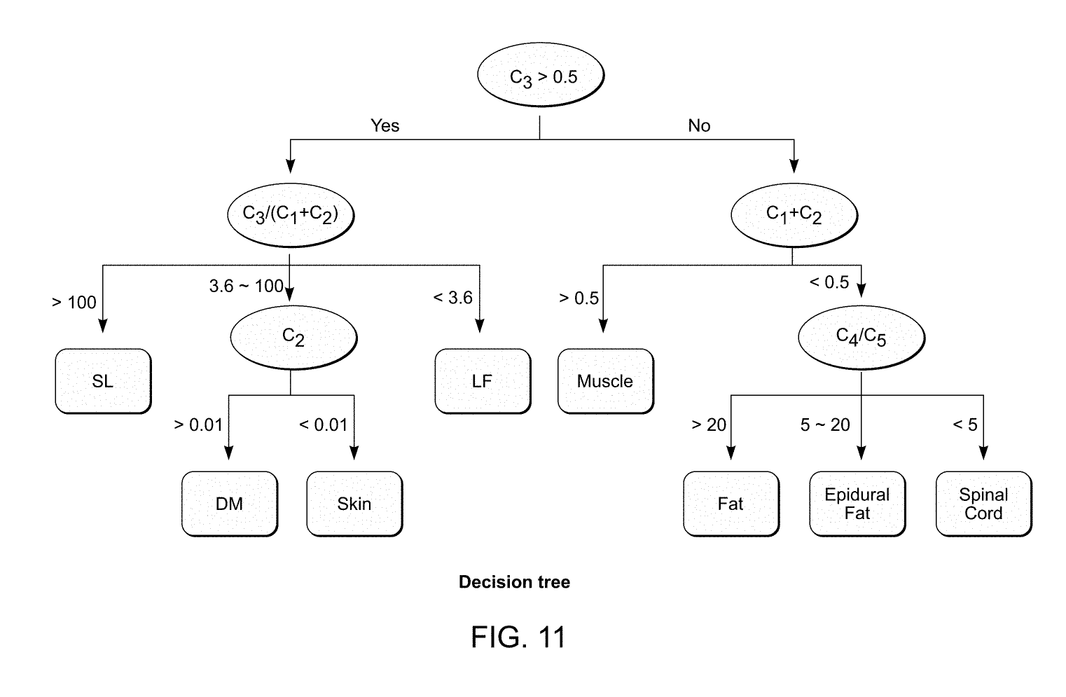

A decision algorithm based on fitting coefficients is used (FIG. 11). As a first step, measurement was divided into two groups based on collagen contents (C₃). The first group (high collagen) includes supraspinous ligament, dura mater, skin and ligamentum flavum, and this group is further categorized based on the relative ratio between actin, albumin and collagen. From the second group (low collagen), muscle can be easily differentiated by its large amounts of actin and albumin. The remaining three groups (fat, epidural fat and spinal cord) are differentiated by their relative ratios of triolein (C₄) and phosphatidylcholine (C₅). Table 4 shows the tissue prediction results. As can be seen, there is no cross confusion and all eight tissue layers can be differentiated.

TABLE 4
Tissue prediction by fitting coefficients-based on decision algorithm.

| | skin | fat | muscle | SL | LF | EF | DM | SC |
|---|---|---|---|---|---|---|---|---|
| Skin | 15 | | | | 2 | | 8 | |
| Fat | | 25 | | | | | | |
| Muscle | | | 25 | | | | | |
| SL | | | | 25 | | | | |
| Ligamentum Flavum | | | | | 25 | | | |
| Epidural Fat | | | | | | 25 | | |
| Dura Mater | | | | 5 | | | 20 | |
| Spinal Cord | | | | | | | | 25 |

Like the traditional loss-of-resistance method, reflection- or image-based methods cannot distinguish among the same type of tissue at different densities. One great advantage of Raman spectroscopy is chemical specificity. Each tissue type has its own signature. Different densities of the same tissue may change the Raman intensity as more or less vibrational bonds will be contained in the sampling volume. However, the spectral shape will not be changed as long as the chemical composition (i.e., tissue type) remains the same. For example, a high-density muscle layer will always have the same muscle spectrum but simply with increased counts. There will never be confusion between muscle and other tissue types (ex. fat or ligament). Therefore, the Raman spectroscopy base method is not affected by a patient's anatomy.

A preferred embodiment of the present invention includes an optical fiber Raman probe which removes the optical fiber background, limits the length of the rigid distal tip to less than a few mm and the diameter to about two mm, for example, to facilitate use in coronary artery catheterization, employs 830 nm excitation, and maximizes signal collection from diffuse sources in order to allow data collection times of a few seconds or less.

A preferred embodiment includes a rod and tube configuration in which the rod and tube of optical filter modules are coated separately, which is easier than coating a single disc having two separate coatings: one in the center to filter the excitation light and one at the edges to filter the collected light. A two-tone disc is preferable to coating a single disc because it is difficult to deposit concentric coatings on a small diameter with a smooth circular interface without gaps or overlapping regions. Each filter can include a stack of dielectric thin films. Such thin film filters can be fabricated by Research Electro-Optics Inc., Boulder, Colo.

FIGS. 12A-12C show longitudinal and transverse views of preferred embodiments of an apparatus including a Raman probe. These embodiments can be inserted within needle probes or the distal ends thereof can be configured with a needle-tip shape. The apparatus 70 includes a two-piece, multiple (for example, dual) wavelength micro-optical dielectric filter module for minimizing and preferably eliminating fiber Raman background in the delivery and collection fibers. This module consists of a rod 82 carrying the excitation dielectric filter coating on one plane face, fitted into the tube 78 carrying the collection dielectric coating on one plane face of the tube. Rods and tubes are used in the embodiment that are made of either sapphire or fused silica which are separately coated with their respective filters prior to assembly. The rod is wrapped or coated with a thin sheet of metal 80 to provide optical isolation between the components. The module is then placed at the distal end of the probe between the fiber bundles and a lens system for collimating the light beams having a lens 86 such as, for example, a ball lens. The lens collects light from high angles and a large area effectively overlapping excitation and collection regions. The ball lens can be fabricated and supplied by Edmund Industrial Optics, New Jersey. In a preferred embodiment, sapphire lenses that are coated with anti-reflection coatings and having an appropriate index for angular acceptance, for example, 1.77 are fabricated by MK Photonics, Albuquerque, N. Mex. Although it is expensive to obtain high quality interference filters at this scale, the cost of the filters is independent of the number of pieces coated; thus, it is possible to coat many filters at once thereby reducing the construction cost of each probe. Furthermore, through additional coating runs, the filter size can be adjusted to create smaller diameter probes for various applications. In a preferred embodiment, the filters are deposited on sapphire or quartz rods and tubes for proper registration with fibers. According to various embodiments, the apparatus may include a fluid channel 95 that can carry fluid between the proximal end and the distal end of the apparatus. The fluid may enter or exit the apparatus through the fluid outlet 91.

FIG. 12C shows a longitudinal view of an alternate preferred embodiment having a paraboloidal mirror disposed in the lens system. The collection angle can be in the range of 0 to approximately 55° with a collection diameter of approximately 1 mm. The paraboloidal mirror collects light from a wider angle and a larger area. According to various embodiments, the apparatus may include a fluid channel 95 that can carry fluid between the proximal end and the distal end of the apparatus. The fluid may enter or exit the apparatus through the fluid outlet 91.

In accordance with preferred embodiments, the choices of fiber diameter and numerical aperture (NA) are dictated by the following considerations. For example, the fiber Raman signal (produces unwanted background) is proportional to the square of the NA and independent of the fiber diameter. Further, low NA is better, and diameter has no effect.

For the excitation fiber, using a lower NA fiber is useful; however, there are issues to contend with. At the input end, low NA makes coupling the energy into the fiber more difficult. In a preferred embodiment, when exciting with a laser with a low beam divergence, reasonable care in mounting the fiber and the matching optics avoids this problem. At the output end, the beam is more confined. This makes the filter construction simpler and more efficient, but illuminating a larger area in order to minimize the potential of tissue damage due to confining the power of the incident beam to a smaller area (spot) can also be important. However, even a smaller diameter spot of laser excitation light incident on the tissue spreads to cover a larger area (typically 0.5-1 mm diameter) because of the aforementioned elastic scattering turbidity, thus mitigating this consideration. In a preferred embodiment, a larger diameter fiber or a distributed array of smaller fibers is used. Preferred embodiments balance the fact that low NA fibers typically exhibit an increased spectral background caused by dopants used in the core and cladding of the fiber to reduce the NA, and hence, use a modest core size and NA for the excitation fiber.

For the collection fibers, the situation is different. The Raman energy collected is proportional to the square of the NA. Therefore, from a signal-to-background perspective, there is an advantage in using high NA collection fibers the size of which is limited by the spectrograph NA. Here, the best choice of fiber NA and fiber diameter is determined by the spectrometer NA, the desired spectral resolution, and considerations of matching optics, as well as the limitation set by filter acceptance angle. In a preferred geometry, one or a few number of delivery fibers are used as the energy of the laser source can be efficiently coupled into the delivery fiber/fibers. However, a greater number of collection fibers is important to increase the area of collection as shown in FIG. 12B. The area for collection is maximized since it is important to optimize collection of Raman light. Taking all these considerations into account, it is best to use as much of the available cross-sectional area of the optical fiber probe for collection fibers, keeping the number and diameter of the delivery fiber(s) to a minimum.

Preferred embodiments include the following trade-offs. For the spectrometer chosen, the desired resolution determines a slit width. Considering the throughput theorem, the requirement on the collection fibers is that the product of fiber NA and diameter equal the product of spectrometer NA and slit width. If it is possible to choose a fiber which satisfies the stronger condition that the fiber diameter equals the slit width and the fiber NA equals the spectrometer NA, the necessity of using matching optics is eliminated and the probe can be directly coupled into the spectrometer. If only the product requirement can be satisfied, then matching optics are needed. At the output end, the collection fibers are arranged in a straight line, which is imaged onto the entrance slit by the matching optics. Occasionally spectrometers use curved slits; the output end of the collection fibers can be modified to match any slit shape. An upper limit on the number of collection fibers is that the height of the fiber array image be less than the slit height or CCD chip, whichever is less. However a smaller limitation may be set by the space available in the collection tip.

In a preferred embodiment, the fiber section of the probe includes a single central excitation fiber with an NA of 0.22 and a core diameter of 200 μm. The buffer of the fiber is matched to the diameter of the excitation filter rod to facilitate proper fiber/filter registration, and has an aluminum jacket to provide optical isolation from the collection fibers. The 200 μm core diameter collection fibers are arranged in two different geometries in two alternate embodiments. The first embodiment consists of two concentric rings of 10 and 17 fibers for the inner and outer ring, respectively. The second embodiment has a single ring of 15 collection fibers. Although the second design has a slightly reduced collection efficiency, it is more flexible and still able to collect a high SNR spectra in short exposure times. The collection fibers all have an NA of 0.26 so that they are f/#-matched to the spectrograph for optimal throughput as illustrated in FIGS. 12A-12C. The diameter of the probe, in a preferred embodiment is less than 2 mm for access to coronary arteries.

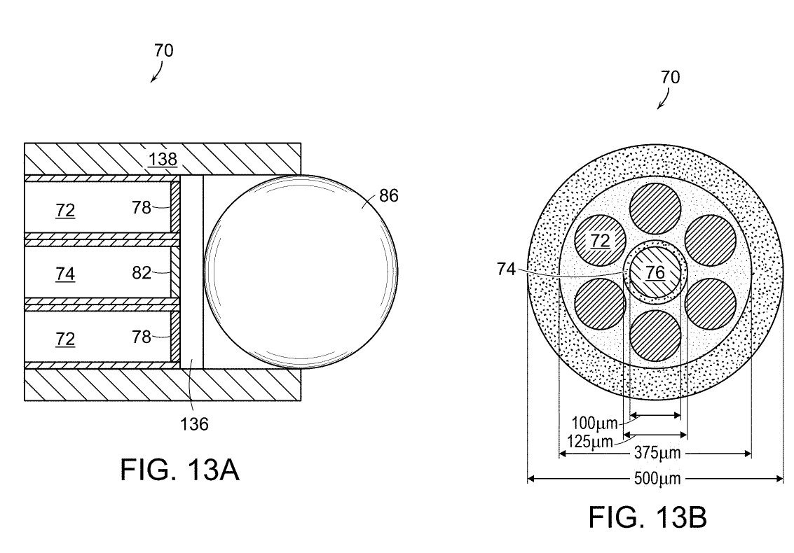

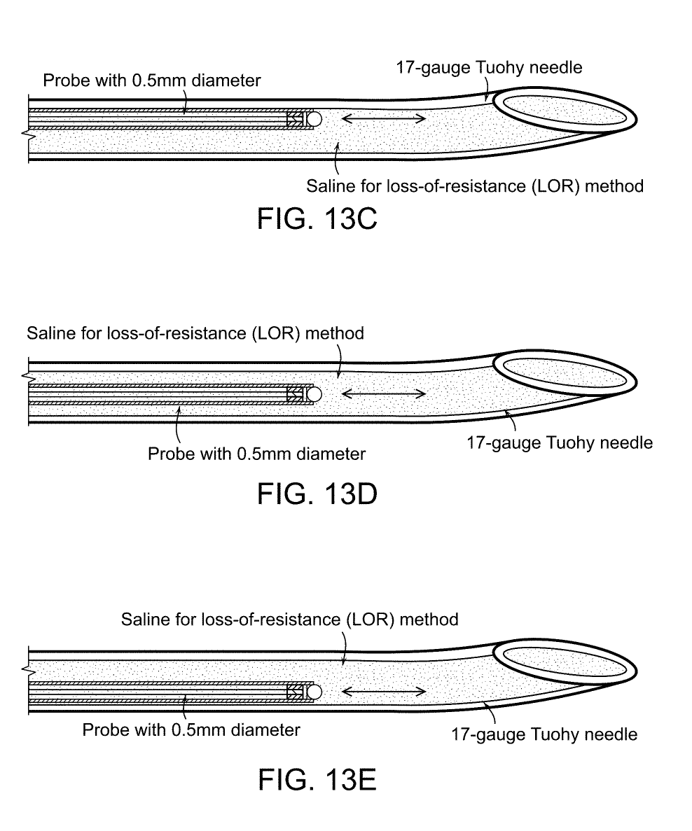

FIGS. 13A and 13B illustrate side and end views of the distal end of a fiber optic probe 70 according to various embodiments. The probe 70 may comprise a metal sheath 138 to protect the components from environmental conditions. In accordance with various embodiments, the probe 70 can contain one or more deliver fibers 74 and one or more collection fibers 72. According to various embodiments, the tips of the fibers may be coated with short-pass filters 82 or long-pass filters 78 as required by the application. A sapphire window 136 may be placed near the distal end to improve imaging and seal the fibers away from the environment. A ball lens 86 may be used to focus light exiting the delivery fiber 74 and gather light at high input angles to be delivered to the collection fibers 72. The fibers may be sheathed in a metal coating 74 to prevent cross-talk between fibers. In certain embodiments, this metal coating 74 may be aluminum. In a preferred embodiment, the diameter of the probe 70 is less than or equal to 500 μm.

FIGS. 13C-E illustrate how the probe is positioned with a needle. In accordance with various embodiments, the needle may be a 17-gauge Tuohy needle and the probe may have a diameter of 0.5 mm. According to various embodiments, the probe may be positioned against a wall or it may be positioned away from a wall of the needle. The probe may be placed at any distance from the distal tip of the needle that satisfies the specific light throughput requirements of the application.

A preferred embodiment provides flexibility with respect to the particular choice of optics for high-throughput collection so that a variety of optical elements can be used to collect the desired AΩ-product. In a preferred embodiment, a ball lens provides highly efficient collection for front viewing optical fiber probes that closely match calculated collection over a radius of 0.35 mm for blood tissue (0.4 mm for artery tissue) while still collecting over large angles. Collection efficiencies greater than 30% are achieved if a small space is maintained between the sample and lens, greater than 10% when in contact with tissue, the likely and more reproducible in-vivo geometry.

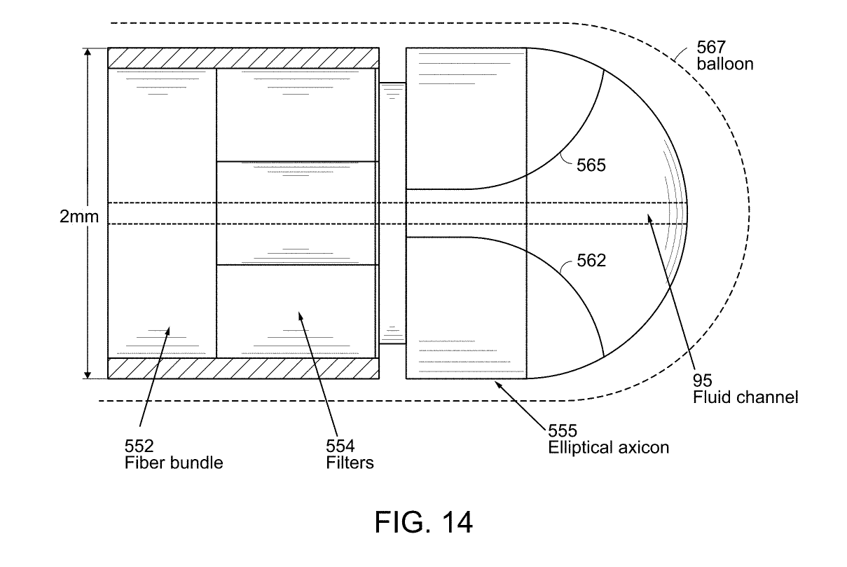

FIG. 14 illustrates a longitudinal view of an alternate preferred embodiment of the side-viewing probe for measuring tissue in accordance with a system of the present invention. The embodiment includes a modified axicon in which the surfaces of the angled sides are made elliptical. FIG. 14 is a preferred alternate embodiments including at least two different radii of curvature on the angled surface to provide circumferential imaging. Circumferential imaging can be obtained in an embodiment by providing beams ranging from approximately 45°-90° angle and rotating the probe to get a circumferential image. Alternatively, delivery fibers can provide light to the tissue and image, such as, for example, six images are collected in collection fibers to get a circumferential image. In one preferred embodiment, the volume between the filters and the angled portion of the axicon comprises solid glass, preferably sapphire wherein the redirection of light occurs via total internal reflection. According to various embodiments, the probe may include a fluid channel 95 that passes through it to carry fluid between the proximal end and the distal end of the apparatus.

In the alternate preferred embodiment as illustrated in FIG. 14, the angled surfaces of the axicon are mirrored which allow for reflections. The laser light is directed radially or non-axially onto the tissue. Further, the surface is elliptical and fabricated using sapphire. The volume between the filters and the axicon may either be filled or empty. The foci of the axicon can be adjusted. The rod-in-tube geometry of filters described in previous embodiments are modified to a tube-in-tube geometry, i.e., a central tube for the excitation filters with a hole in the middle for the central channel and an outer tube for the collection filters.

Of the three spectral signals (Raman, DRS and fluorescence), Raman is typically the weakest. Thus, a spectral probe capable of collecting high-quality Raman spectra can collect fluorescence and reflectance spectra as well. The spectral probe design for the combined instrument is single-ring front-viewing Raman probe.

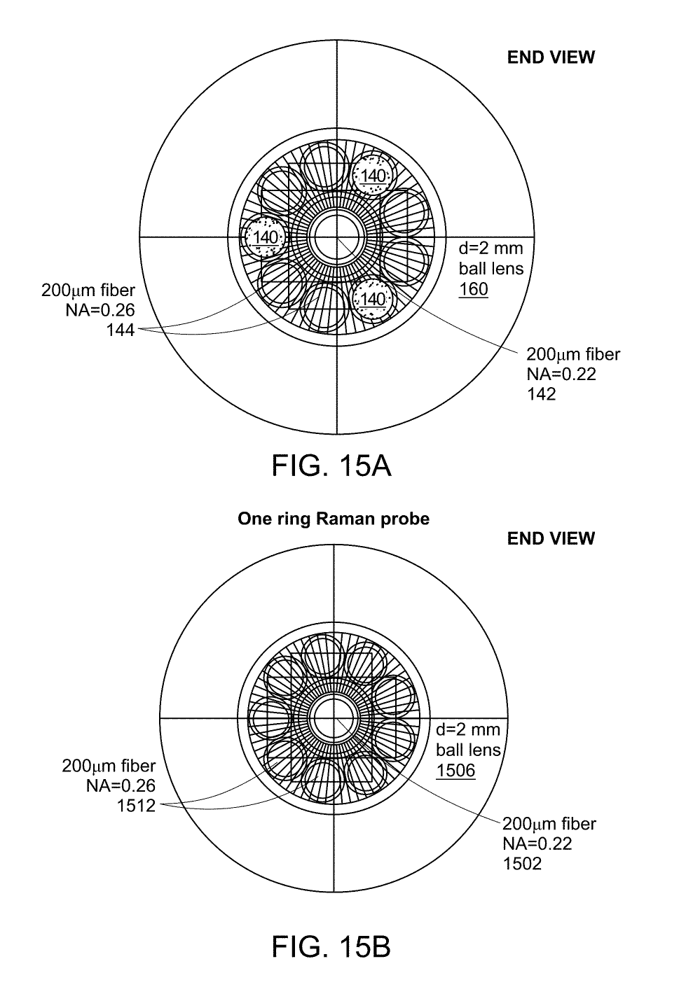

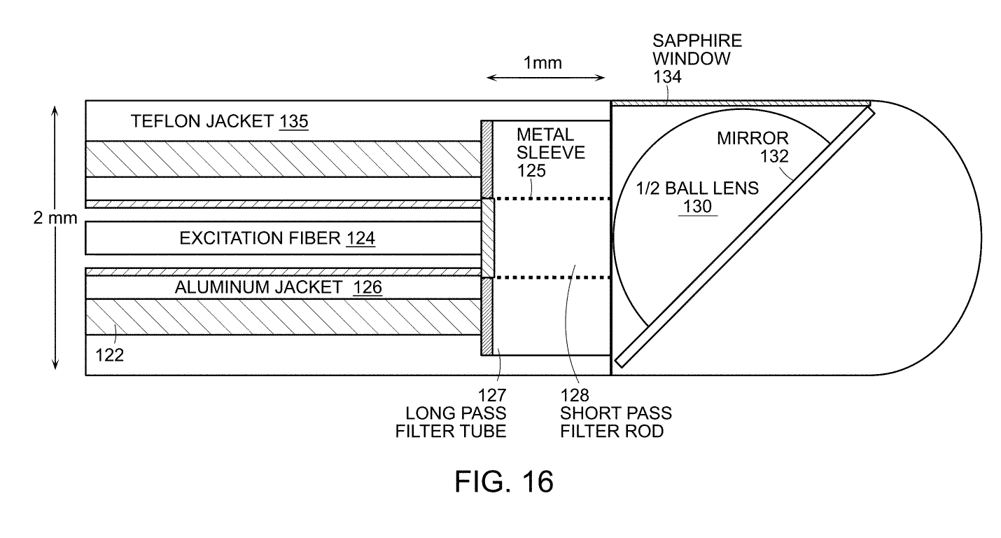

Placement of filters and ball lens can be the same as the Raman probe, but the filter characteristics have tighter specifications when used with all three spectral modalities. An exemplary embodiment comprises a reduced diameter 9-around-1 probe 100 and excitation/collection trajectories through a ball lens 106 that contacts tissue 108. Similar to the Raman probes, the filter module has a filter rod 104 placed on the delivery fiber with transmittance from 300-830 nm and no transmittance (<1%) beyond 850 nm. A filter tube placed on the collection fibers has transmittance from 300-810 nm and from 850-1000 nm and with a narrow 40 nm band centered at 830 nm having low transmittance. An end view of the probe is shown in FIG. 15A with collection fibers 112 positioned in a circular array around central excitation fiber 102. A side looking probe 120 is shown in FIG. 16 in which a half ball lens 130 is in contact with a mirror 132 to reflect light from excitation fiber 124 and filter rod 128 through sapphire window 134. Light returning from the tissue is reflected into collection fibers 122 through long pass filter tube 127. A metal sleeve 125 surrounds filter 128. An aluminum jacket surrounds the excitation fiber 126. A Teflon jacket 135 provides the cylindrical tube that forms the outer wall of the catheter.

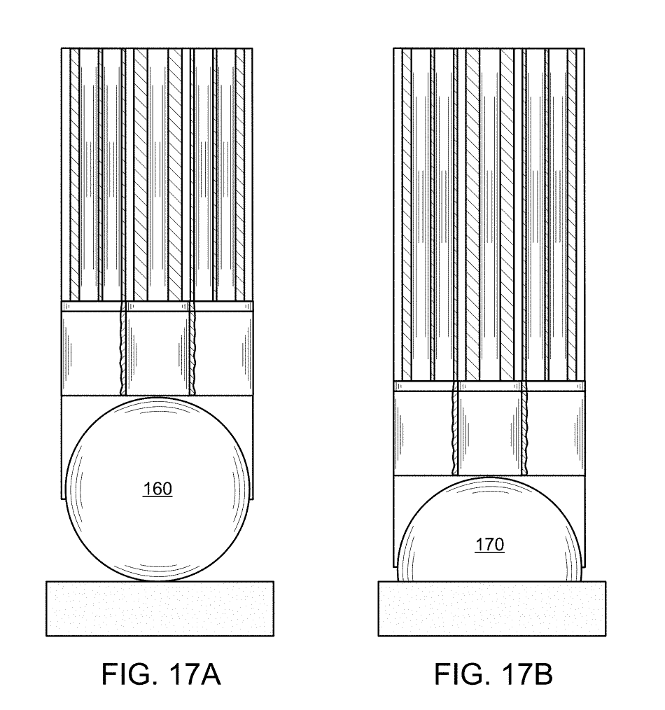

FIG. 15A illustrates an end view of a design in which a first group of 3 collection fibers 140 are used to collect reflected light and 3 pairs of fibers 144 collect the Raman light passing through ball lens 160. The central fiber 142 directs light through the forward looking probe with lens 160 in FIG. 17A or the half-ball lens 170 of FIG. 17B.

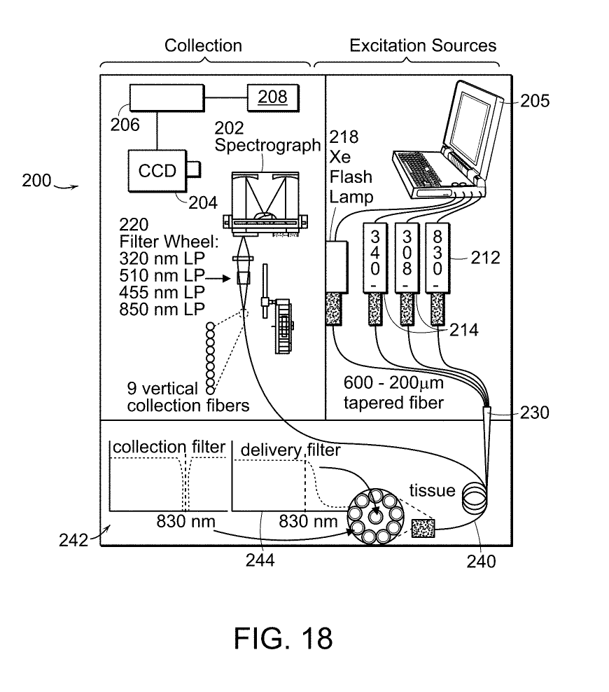

A compact portable MMS instrument that incorporates all three spectroscopic modalities (DRS, IFS and Raman) is shown in FIG. 18. The fourth modality, LSS, requires no extra instrumentation. A preferred MMS instrument 200 uses solid state light emitting diodes to reduce the instrument size, complexity and cost, and to eliminate many maintenance issues related to excimer laser and dye cell operation. The MMS instrument can employ a common spectrograph 202 and CCD 204 for all spectral acquisition.

To accommodate the requirements for using all three spectroscopic modalities, spectra are collected over the wavelength range 300-1000 nm. Excitation light for each modality is delivered sequentially to the sample, and fluorescence, DRS and Raman spectra are acquired. This is followed by real-time analysis of the data, during which IFS spectra are derived from the fluorescence and DRS spectra. The information from the different modalities provides depth-sensitive complementary chemical and morphological information on tissue sites.

The measurements include IFS spectra excited at 308 and 340 nm, DRS and Raman spectra. The combined TMS/Raman instrument is used for FastEEM fluorescence excitation wavelengths to determine the diagnostic value of the various excitation wavelengths. The most appropriate two or three fluorescence wavelengths can be used in the integrated system.

Data acquisition, analysis and tissue characterization preferably occurs in 5 sec or less. Triggering of the light sources is accomplished by means of a National Instruments Timer/Counter card and a Princeton Instruments CCD controller, respectively. The sequence of operation can be controlled by computer 205 as follows: (1) Initialize CCD for spectral acquisition; (2) open shutter for the CCD and activate insertion of appropriate collection filter; (3) trigger light source (LED, diode laser or flashlamp); (4) acquire spectrum; (5) close 10 shutter; (6) read/transfer data and store in computer 206 and display at 208. The time for acquiring all spectra depends upon the excitation power; thus, the exposure time can be adjusted to accommodate signal levels.

Separate excitation and reflectance sources can be used for each spectroscopic modality. Laser emitting diodes 214 (˜1 mW) provide fluorescence excitation light at 308 and 340 nm, a 60 W xenon flashlamp generates a continuous spectrum from 300-1000 nm for DRS, and a laser diode 212 at 830 nm (500 mW) will generate the Raman excitation light. A flashlamp 218 can be used in the FastEEM, and the 830 nm laser diode in the Raman system. Each of these four light sources can be focused onto separate 200 μm core diameter optical fibers, and then coupled together into a 600-to-200 μm tapered optical fiber The output can be connected to the combined spectral probe via an SMA connector. The system enables fluorescence excitation wavelengths to be added and/or changed.

UV diode sources can be used as compact light sources in the 300-340 nm range. UV light emitting diodes at wavelengths as short as 275 nm or UV LEDs in the 305-360 nm wavelength range can be used. Current 308 nm LEDs produce 1-2 mW of CW power in a bandwidth of 10-15 nm, emitted from a 0.1 mm aperture over a 30° angular range. Because of this large bandwidth, a filter can be used to restrict the light to a 2 nm bandwidth. Thus, under present conditions, −1 μJ of 308 nm light can be delivered via 200 micron core, 0.26 NA, fused silica optical fiber in 10 ms, resulting in the acquisition of high SNR fluorescence spectra. Characteristics of 340 nm LEDs are even more favorable.

Each of the spectral probe collection fibers, typically nine, (fifteen in one design) are coupled to an SMA connector mounted on the front panel of the instrument. Long (wavelength) pass filters 220 mounted on a programmable wheel driven by a stepper motor are positioned in the return beam path to prevent Raman and fluorescence excitation light scattered from the tissue from entering the spectrograph. Since the reflectance measurements cover a broad range (300-1000 nm), the acquired spectra contain second order contributions. Taking two reflectance measurements, one with no filter and another with a long pass 500 nm cutoff filter (mounted on the wheel), eliminates these contributions. The unfiltered reflectance provides spectral information below 600 nm, and the filtered reflectance provides information above 500 nm. The Princeton Instruments Spec10:400 BR CCD camera of the Raman system can be coupled to an Acton Research Spectra Pro 150 spectrograph with a grating blazed at 500 nm and 200 grooves/mm. Alternatively two separate gratings or dispersive elements can deliver different light modalities onto separate regions of the detector.

This combination of fluorescence, reflectance and Raman capabilities in one instrument provides a compact clinical instrument. With a single spectrograph/CCD combination, a spectral range of 300-1000 nm is covered compared to 155 nm in our existing Raman system. This causes an increase in spectral dispersion by a factor of 4.5, and a reduction in system resolution from 10 to 45 cm⁻¹. However, if the spectral resolution degrades the accuracy of the Raman fit coefficients significantly such that diagnostic accuracy is also degraded. A two spectrograph/CCD system can also be used with one spectrograph/CCD combination dedicated to Raman while the other is dedicated to fluorescence/reflectance. A high-speed mirror will direct the collected light to the appropriate spectrograph/CCD combination.

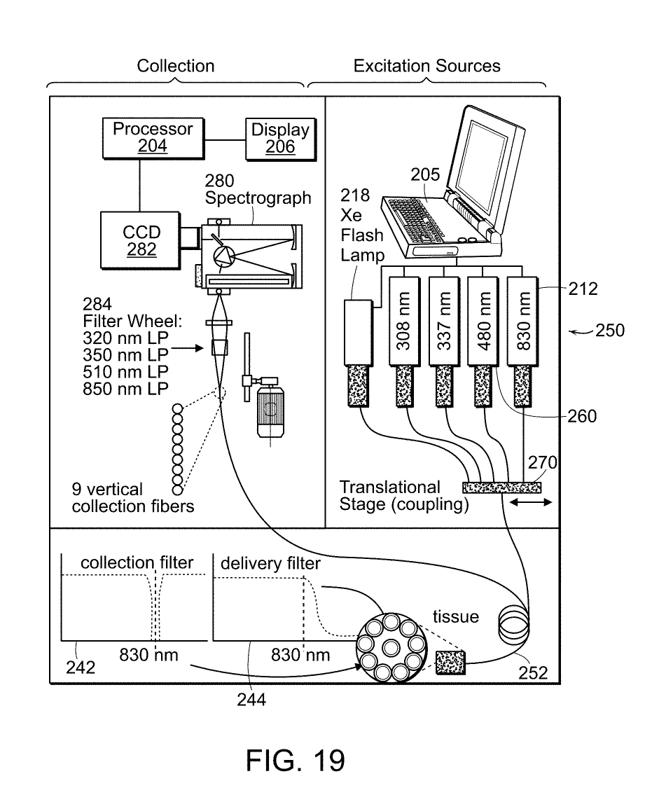

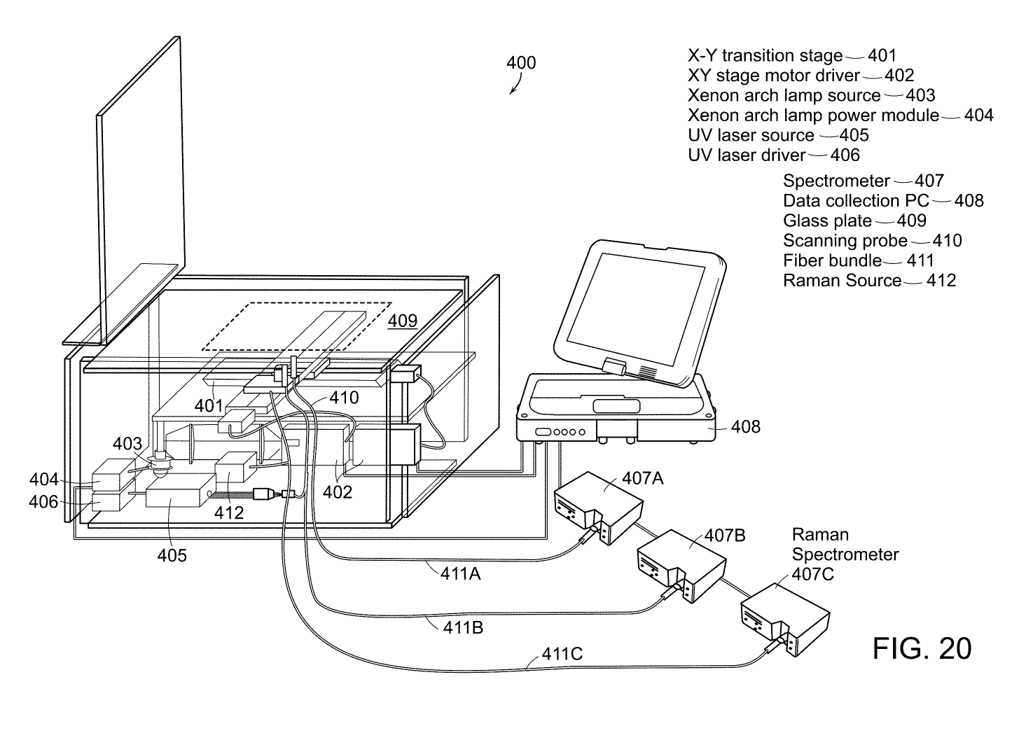

A further embodiment of a system 250 is shown in FIG. 19 in which a translational stage 270 is used to couple light from the source sequentially into the probe 252. This contrasts with the prior embodiment where the sources are coupled to probe 240 with combiner 230 to provide simultaneous illumination. The delivery 244 and collection 242 filters are shown schematically. Another source 260 is also used and accounted for in the filter wheel 284, spectrograph 280 and detector 282 system.

FIG. 20 shows a schematic diagram of a tissue scanner system 400 in accordance with the invention. The scanner employs a unitary multimodal optical fiber probe 410, or probes, that can be employed in a multimodal clinical spectroscopy system for point spectroscopy measurements. Two (or three) optical fiber probes can also be used, one for DRS, another for IFS (and optionally, a third for Raman measurements), at a fixed separation of 0.75 cm to minimize cross talk between the probes. For the single probe system, a probe shown in FIG. 20 using a single light delivery fiber 142, and pairs of separate collection fibers 140, 144 and 146 to collect DRS, IFS and Raman spectra, respectively. This can use a distal filter and lens system as described previously herein with the distal filters configured for each collection frequency range of the different modes. Further details regarding the multimode system are provided in U.S. application Ser. No. 13/338,920 filed on Dec. 28, 2011, the entire contents of which is incorporated herein by reference.

Each probe can include a fiber bundle with a single central fiber that delivers excitation light to the tissue, surrounded by a ring with a plurality of optical fibers that collect reflected and fluorescent light returning from the sample and transmit it to the spectrograph (all fibers have 200 μm core and NA=0.22) terminated with a transparent, protective optical shield. A 75 W Xenon arc lamp 403 (Oriel Instrument, USA) and power module 404 can be used to generate excitation light for DRS and a 7 mW Q-switched solid state laser 405 at 355 nm (SNV-40E-000, Teem Photonics) with driver circuit 406 is used to generate excitation light for IFS. Other excitation wavelengths can be used for other diagnostic applications. An infrared Raman source 412 can be used for Raman spectral measurements. Signals are collected with miniature spectrometers (USB2000+, Ocean Optics). The spectrometers 407A, 407B, 407C have spectral resolution of 2 nm at full width half maximum (FWHM). The collection fibers 411A, 411B, and 411C are coupled to the corresponding reflectance, fluorescence and Raman spectrometers. The wide area imaging capability is achieved in an inverted geometry through a standard glass plate 409 (10×12×1; 16 inches) on which the specimen rests. There is no interference from glass fluorescence with the biomolecular fluorophores of interest, such as collagen and NADH.

The glass plate flattens the tissue surface and provides a reasonably uniform probe-tissue imaging distance. This provides for quantitative measurements, by preserving the key optical characteristics of the probe 410 (spot size and NA), and takes full advantage of probe-based spectroscopic models. The portable device measures 2×1×1 ft, weighs 30 lbs or less and can easily fit in most clinical spaces including patient examination rooms, procedure rooms and operating rooms.

DRS, fluorescence spectra (350-700 nm), and Raman data can be obtained for each spot scanned. After background subtraction and normalization with 20% Spectralon white reflectance standards (Labsphere, NH), DRS spectra are analyzed using a mathematical model based on the diffusion approximation of light propagation in tissue. IFS spectra are then obtained, by correcting the raw fluorescence spectra for the effects of tissue absorption and scattering using the corresponding DRS spectra, and analyzed with data processor 408 using a linear combination model based on multivariate curve resolution (MCR), a standard chemometric method. Spectral modeling provides physically meaningful fitting parameters that are quantitative measures of the contributions of specific tissue components. These spectral parameters are the basis of decision algorithms used in the diagnosis of breast and other cancers. DRS modeling yields 3 scattering parameters: A, which is related to the amount of Mie scatterers; B, which is related to the size of the scatterers; and C, which is related to the amount of Rayleigh scatterers; and absorption fitting parameters for hemoglobin (Hb) and beta-carotene, two well-characterized absorbers in breast tissue. IFS modeling yields fluorescence fitting parameters related to NADH, a cellular metabolite, and collagen, a fluorophore that is more abundant in the fibrous stroma of breast cancer than in normal breast tissue. Such Raman probes or multimodal probes as described herein can be used to guide sample selection for tissue biopsies of many organs including the lung, brain, breast, bone marrow, liver, kidney, bowel, muscle, spleen, pancreas, prostrate and lymphnodes. The basis spectra coefficients are selected based on the particular tissue of interest and can further include features of the particular pathology to be diagnosed (e.g. cancer).

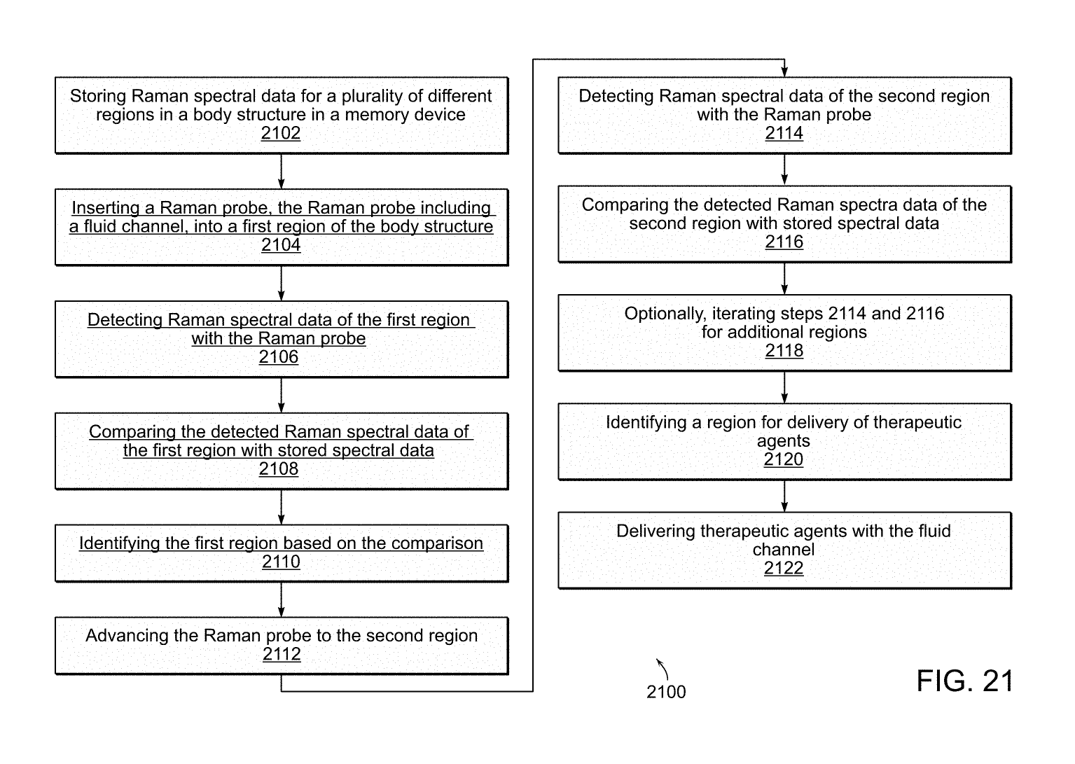

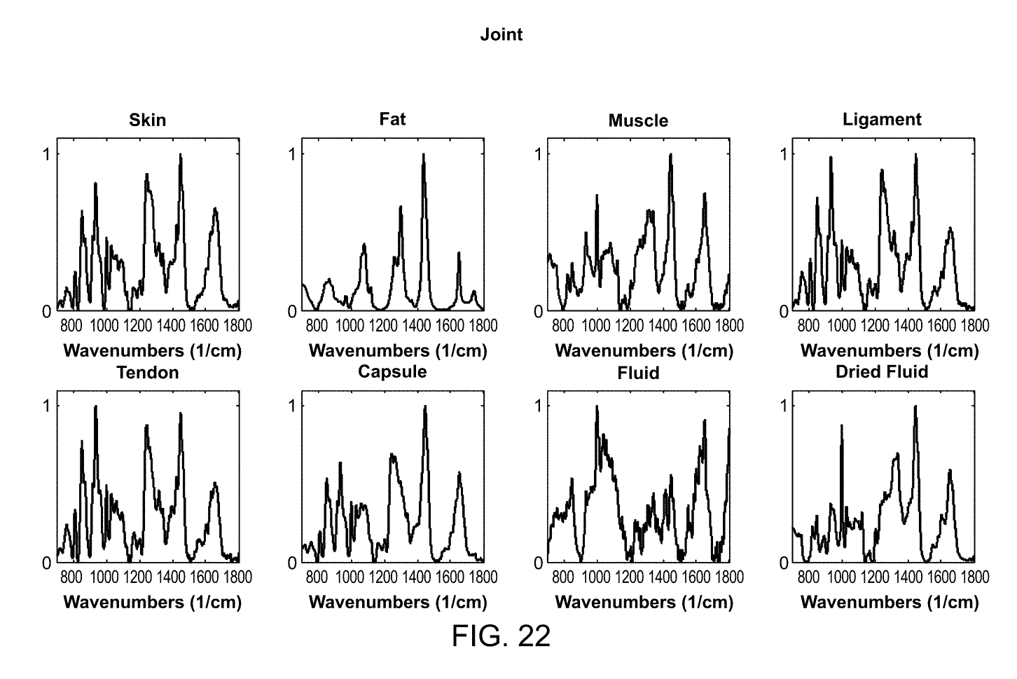

FIG. 21 illustrates a method 2100 for guiding delivery of a probe through a body structure to deliver a therapeutic agent. The method 2100 includes a step of storing 2102 Raman spectral data for a plurality of different regions in a body structure in a memory device. The method 2100 includes a step of inserting 2104 a Raman probe, the Raman probe including a fluid channel, into a first region of the body structure. The method 2100 includes a step of detecting 2106 Raman spectral data of the first region with the Raman probe. The method 2100 includes a step of comparing 2108 the detected Raman spectral data of the first region with stored spectral data. The method 2100 includes a step of identifying 2110 the first region based on the comparison. The method 2100 includes a step of advancing 2112 the Raman probe to a second region. The method 2100 includes a step of detecting 2114 Raman spectra data of the second region with the Raman probe. The method 2100 includes a step of comparing 2116 the detected Raman spectral data of the second region with stored spectral data. The method 2100 includes an optional step of iterating 2118 steps 2114 and 2116 for additional regions. The method 2100 includes a step of identifying 2120 a region for delivery of therapeutic agents. The method 2100 includes a step of deliver 2122 therapeutic agents with the fluid channel.

Animals used for validation in live animal experiments will be similar to those from which tissue was obtained for ex vivo proof of concept experiments, 40-50 kilogram, male and female, 3-4 month old, Yorkshire swine. This species was selected, and is appropriate, because the same tissues are present as in humans and the process of epidural needle and catheter placement in swine resembles that in humans.

A single animal can be anesthetized with five milligrams per kilogram of telazol given intramuscularly. Once telazol has induced general anesthesia, the animals are intubated and ventilated. General anesthesia is maintained with 1-2% inhaled isoflurane gas. Next, an intravenous catheter is placed. The animal remains under general inhalational anesthesia during this time with vital signs monitored and recorded every 15 minutes in order to assess the animal's level of discomfort. The depth of anesthesia can be adjusted as needed to ensure adequate analgesia and anesthesia. Next, the prototype epidural needle fitted with a 0.5 mm diameter spectroscopy probe is inserted at the midline of the posterior thorax between spinous processes and lateral to the midline in order to mimic the two commonly used approaches to epidural space localization with the traditional loss of resistance (LOR) technique. Raman spectroscopy (RS) data is recorded continuously (data acquisition approximately once per second) during epidural needle insertion. The probe-in-needle is advanced at 1 mm increments starting at the skin, through the epidural space, and ending at the spinal cord in order to acquire data from each tissue type along both of the two trajectories. As previously done in swine cadaveric tissue measurement, multiple midline and lateral insertions will be carried out so that a large data pool can be acquired from each live animal. A minimum of five midline and five lateral insertion points will be carried out on each animal. One millimeter increments will be used in order to accurately identify each transition from one tissue type to another as some of the tissue layers are approximately 1 mm thick.

At the completion of the measurement, animals are euthanized with 100 milligrams per kilogram of pentobarbital given intravenously. Immediately following euthanization, cerebral spinal fluid (CSF) is removed from the animal, followed by en block excision of the spinal column corresponding to the lumbar and lower thoracic regions. As done previously, the excised spinal column is dissected to separate tissues of interest. Each sample, including CSF, is scanned with RS. After scanning, each tissue sample is placed in 10 milliliters (mls) of formalin, embedded in paraffin, cut, placed on slides, and stained with hematoxyline and eosin. In order to confirm tissue types, all slides are reviewed, and identified, by a qualified neuropathologist. Lastly, data from the probe-in-needle insertions and from the dissected tissues are compared to previously acquired Raman spectroscopy data.

For Raman spectroscopy for epidural space localization, human tissues can be scanned by RS. Human tissue measurements using tissue from skin to the spinal cord including epidermis/dermis, fat, skeletal muscle, supra-/intraspinous ligament, ligamentum flavum, epidural fat, dura mater, cerebral spinal fluid, and spinal cord can be individually scanned by Raman spectroscopy and compared to previously acquired swine tissue data.

Cadaveric spine tissue can be obtained and an epidural needle fitted with a 0.5 mm diameter spectroscopy probe can be inserted at the midline of the posterior thorax between spinous processes as well as lateral to the midline as in prior swine tissue measurements. Raman spectroscopy (RS) data can be recorded continuously during epidural needle insertion. The probe-in-needle can be advanced at 1 mm increments starting at the skin, through the epidural space, and ending at the spinal cord in order to acquire data from individual tissues along each of the two trajectories. As previously done multiple midline and lateral insertions can be carried out so that a large data pool can be acquired from each human cadaveric spine tissue. A minimum of five midline and five lateral insertion points are used to provide reference spectral data.

The human cadaveric spinal column can be dissected to separate the following tissues: epidermis/dermis, fat, skeletal muscle, supra-/intraspinous ligament, ligamentum flavum, epidural fat, dura mater, and spinal cord. Each sample is scanned with RS. After scanning, each sample is placed in 10 milliliters (mls) of formalin, embedded in paraffin, cut, placed on slides, and stained with hematoxyline and eosin. In order to confirm tissue types, all slides will be reviewed, and identified, by a qualified neuropathologist. Lastly, data from the probe-in-needle insertions and from the dissected tissues are compared to previously acquired reference Raman spectroscopy data from human tissues.

Raman spectroscopy and multi-modal spectroscopy are powerful tools to detect a needle-tip location in laparoscopic and arthroscopic surgery techniques as well as other applications involving transdermal penetration of a plurality of tissue layers. Porcine animals were treated as above. After euthanization, knee joints were excised from the animals. Tissue was stored at 4 degrees Celsius until use and used within 24 hours of euthanization. Tissue was scanned when at room temperature. Individual tissues from the swine knee joints were dissected and excised. With reference to FIG. 22, Raman spectroscopy data were acquired from individually dissected tissues. In an arthroscopic surgery operation, the needle is advanced through layers of skin, fat, muscle, ligament, tendon, and capsule until the tip finally penetrates into the synovial fluid of the joint. Spectra were obtained corresponding to tissue layers of skin, fat, muscle, ligament, tendon, and capsule as well as synovial fluid and dehydrated synovial fluid. Through an appropriate choice of basis spectra, these spectral data can be decomposed and processed to allow detection and differentiation of each layer as the needle tip proceeds. In some embodiments, the probe may be adapted to detect other structures or items present in synovial fluid by viewing through the synovial fluid and compensating for its presence in the measured spectra.

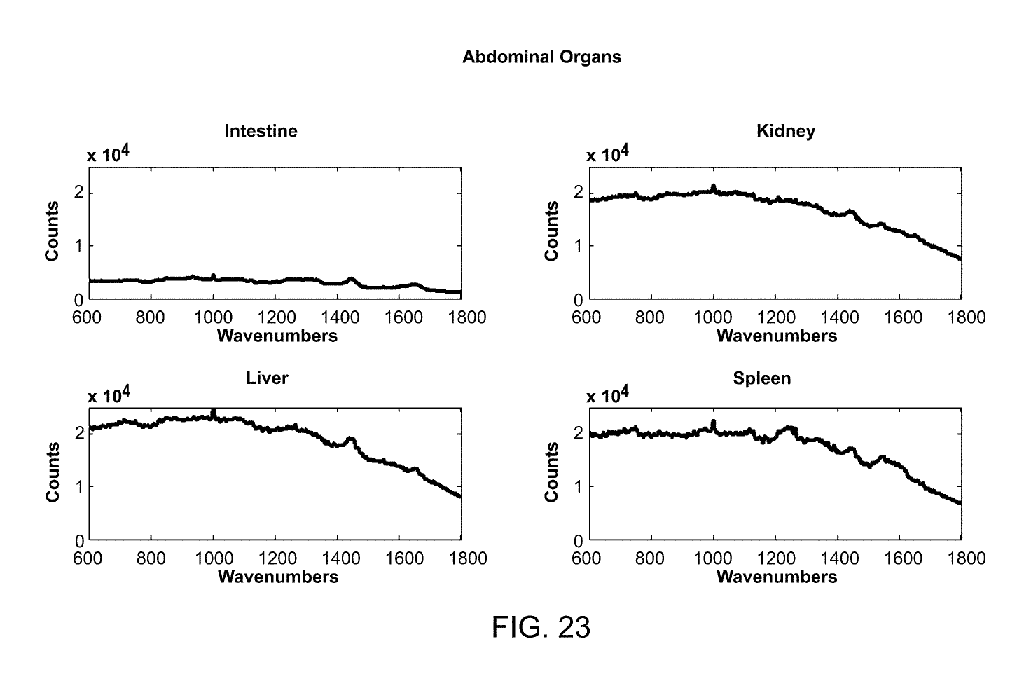

Embodiments of the present disclosure are useful in placing needles or other instruments during laparoscopic procedures. The task of needle placement in laparoscopic surgery is strongly geared towards avoiding advancing the needle into various bodily organs. Using standard laparoscopic techniques, it is often difficult to detect when a sensitive organ has been lacerated or damaged until a time later than the procedure when the patient begins to exhibit symptoms. As described above, Raman or multi-modal spectroscopy can be used to identify tissue layers as a needle or instrument advances into an abdomen. However, multi-modal spectroscopy can also be used to identify events when the needle penetrates into organs or cavities where it was not intended to penetrate. With such knowledge in hand, the practitioner can halt needle advance immediately and take step to remediate the improper needle location before continuing the laparoscopic technique. Turning to FIG. 23, Raman spectroscopy was performed on the tissues of the abdominal cavity. Porcine animals were treated as above. After euthanization, tissue corresponding to the abdominal wall and abdominal organs were excised. Tissue was stored at 4 degrees Celsius until use and used within 24 hours of euthanization. Tissue was scanned when at room temperature. Individual tissues were dissected and scanned. Spectra were acquired corresponding to skin, fat, and muscle from the abdominal wall as well as the intestine, kidney, liver, and spleen. As can be seen, the Raman spectra of intestine, kidney, liver, and spleen are highly distinguishable from the spectra of skin, fat, and muscle (FIGS. 6 and 7).

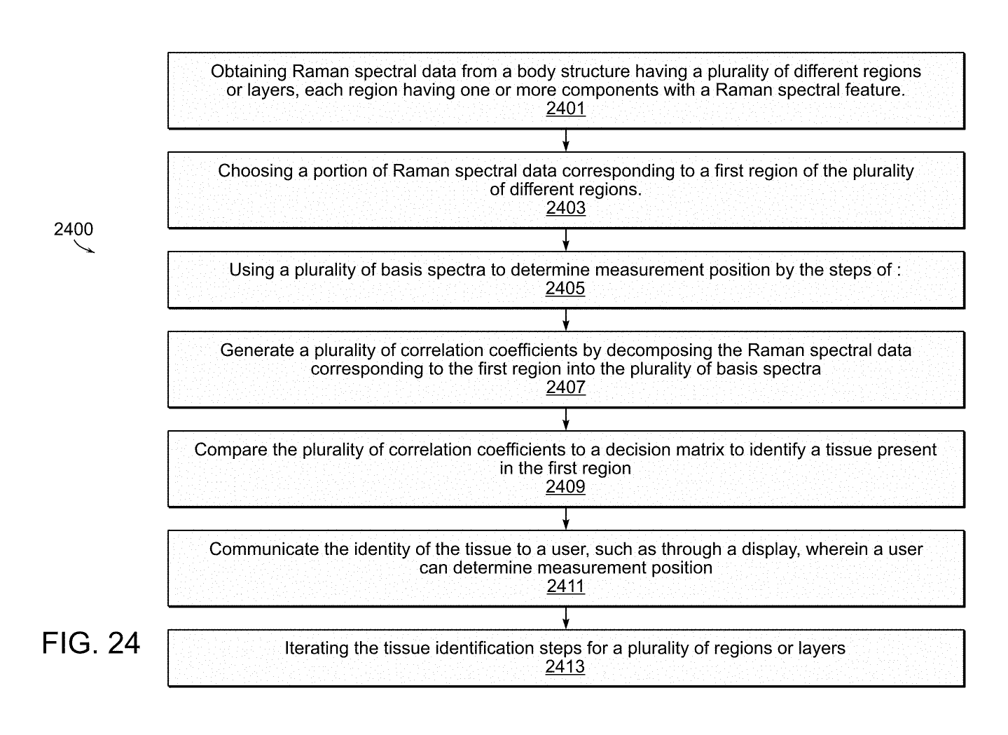

FIG. 24 describes a method 2400 of identifying a tissue according to various embodiments. The method includes using devices for communicating to the user the status of the needle position such as by virtual display of needle location as it pass through layers. The method 2400 includes obtaining 2401 Raman spectral data from a body structure having a plurality of different regions or layers, each region having one or more components with a Raman spectral feature. The method 2400 includes choosing 2403 a portion of Raman spectral data corresponding to a first region of the plurality of different regions. The method 2400 includes using 2405 a plurality of basis spectra to determine measurement position by the following further sub-steps. One sub-step is generating 2407 a plurality of correlation coefficients by decomposing the Raman spectral data corresponding to the first region into the plurality of basis spectra. Another sub-step is comparing 2409 the plurality of correlation coefficients to a decision matrix to identify a tissue present in the first region. A further sub-step is communicating 2411 the identity of the tissue to a user, such as through a display, wherein a user can determine measurement position. The method 2400 also includes iterating 2413 the tissue identification steps for a plurality of regions or layers.

In accordance with various embodiments, the systems and methods of the present disclosure are not limited to use solely by human operators. In some embodiments, a motorized needle injector under feedback control may be used to place a needle tip at a pre-defined body tissue or organ. In a robotic control system, multi-modal spectral measurements are obtained as the motorized needle injector advances toward a target site on the body. The multi-modal spectral measurements are compared to basis spectra contained within a memory of the robotic control system. The robotic control system can use a fast measurement and analysis refresh rate to obtain information about the location of the needle tip in nearly continuous time. In an exemplary embodiment, the robotic control system contains a master override switch that, when activated, can cause withdrawal of the needle from a patient in a quick but controlled manner.

In various embodiments, the probe may be used in conjunction with a data processor, memory, and display. When a tissue has been positively identified as described above, the display may use that information to show an indicator to the user of the location of the needle tip. For example, the display may show a schematic of the body system being penetrated by the needle tip and may indicate on the schematic where the needle tip is located. As another example, the display may show the name of the tissue wherein the needle tip is presently located. In some embodiments, the display may show a warning when a tissue has been identified that was not intended to be penetrated. For example, the display may show a warning if the needle tip has penetrated a vital organ such as a stomach, kidney, or intestine while the practitioner is performing a laparoscopy operation.

While the present inventive concepts have been described with reference to particular embodiments, those of ordinary skill in the art will appreciate that various substitutions and/or other alterations may be made to the embodiments without departing from the spirit of the present inventive concepts. Accordingly, the foregoing description is meant to be exemplary and does not limit the scope of the present inventive concepts.

A number of examples have been described herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

1. A needle probe system to identify a plurality of tissues comprising:
a fiber optic device including at least one light delivery and collection optical fiber;
an optical beamshaping element positioned at the distal end of the fiber optic device; and
a needle having a fluid channel, the fiber optic device being positioned to deliver and collect light at a distal end of the needle.

2. The system of claim 1 wherein the needle has a curved outer surface or comprises a Tuohy needle.

3. (canceled)

4. The system of claim 1 further comprising a laser light source and at least one optical filter disposed at the distal end of the fiber optic device.

5. The system of claim 4 further comprising a detector, a data processor, and a memory device that stores reference data, the reference data including basis spectra such that the processor analyzes data for a plurality of different body regions.

6. The system of claim 1 wherein the fluid channel is coupled to a manual pressure device to perform loss-of-resistance needle-tip placement.

7. The system of claim 1 wherein at least one light collecting fiber receives light from a direction at an angle to the curved outer surface.

8. (canceled)

9. The system of claim 5, wherein the body regions comprise different layers of tissue.

10. The system of claim 9 wherein the different layers of tissue cover a spine, an abdomen or a joint.

11. The system of claim 5 wherein the basis spectra contain one or more components with a Raman spectral feature.

12. The system of claim 1 further comprising a probe fluid channel within the probe to deliver a therapeutic agent.

13. The system of claim 1 further comprising an epidural syringe.

14. The system of claim 1 further comprising a stylette that contains a lumen adapted to pass the fiber optic device.

15. The system of claim 1 further comprising a coupling device having an attachment lip that hooks over a bevel of the needle to attach the beamshaping element.

16. The system of claim 1 wherein the needle comprises a fluid opening and an optical aperture at different positions on the distal end of the needle.

17. The system of claim 1 wherein the optical beamshaping element comprises at least a portion of a ball lens or an ellipsoidal mirror.

18. (canceled)

19. The system of claim 1 wherein the needle comprises protrusions along an interior wall and the fiber optic probe comprises notches along an exterior wall, the notches and protrusions adapted to mate to position the fiber optic probe with respect to the needle.

20. (canceled)

21. The system of claim 9, wherein the different layers of tissue comprise two or more of dermal tissue, adipose tissue, skeletal muscle, supra-/intra-spinous ligament, ligamentum flavum, dura mater, epidural fat, intestine, kidney, liver, spleen tissue, ligament, tendon, capsule, synovial fluid, desiccated synovial fluid, or spinal cord tissue.

22-23. (canceled)

24. The system of claim 1, further comprising a tube or catheter for delivery of a therapeutic agent.

25. The system of claim 5, wherein each of the basis spectra correspond to one or more of actin, albumin, collagen, triolein, or phosphatidylcholine.

26. The system of claim 5, wherein the data processor decomposes the collected light according to the basis spectra stored in the memory device to define a plurality of correlation coefficients that are compared to a decision matrix to identify a source of the collected light.

27. (canceled)

28. The system of claim 5, further comprising a display that displays measurement position information that a user can use to determine needle tip placement and displays information that warns a user that the needle tip is misplaced.

29. The system of claim 1, wherein a detection zone of the fiber optic device is less than 2 mm from the distal end of the needle.

30. The system of claim 1, wherein the fiber optic device is less than 1 mm in diameter and comprises a single sapphire fiber.

31. The system of claim 1 wherein the fiber optic device fits within a spring-loaded blunt catheter and a proximal end is optically coupled to a filter and a spectrometer.

32. A method of identifying a tissue comprising:
processing, with a data processor, Raman spectral data in a first region of the plurality of different regions using a plurality of basis spectra;
decomposing the Raman spectral data into a plurality of correlation coefficients representing the first region into the plurality of basis spectra;
comparing the plurality of correlation coefficients to a decision matrix to identify a tissue present in the first region; and
recording the identity of the tissue.

33. (canceled)

34. The method of claim 32 further comprising processing at least one of autofluorescence spectral data or reflectance data.

35. (canceled)

36. The method of claim 32 further comprising indicating a measurement position within a body structure on a display and displaying a virtual position of a probe relative to a spine, an abdominal structure, or a joint structure such as a knee, a hip, or a shoulder.

37. (canceled)

38. The method of claim 32 further comprising:
identifying a sequence of tissue layers by processing data with an algorithm to identify the tissue layer using basis spectra and spectral data; and
recording spectral data from a needle probe.

39. The method of claim 38 wherein a layer comprises epidermis or fat or skeletal muscle or ligament or a spinous ligament or a tendon or a joint capsule or an abdominal organ.

40. The method of claim 32 wherein a basis spectrum includes at least actin, albumin, collagen, triolein, or phosphatidylcholine.

41. The method of claim 32 wherein each Raman spectrum has a vector representation, the method further comprising computing a quantitative value for each tissue component.

42-44. (canceled)

45. The method of claim 32 further comprising performing a tissue biopsy or a neural block.

46. The method of claim 32 further comprising a plurality of light sources emitting at different wavelengths that are coupled to a probe having a beam shaping element coupled to a distal end of the needle to illuminate a cutting region.

47. A method for guiding delivery of a probe through a body structure to deliver a therapeutic agent, comprising:
inserting a Raman probe into a body structure having a plurality of different regions, each region having one or more components with a Raman spectral feature;
measuring Raman spectral data in a first region of the plurality of different regions to determine a location of the probe in the first region;
advancing the probe from the first region into a second region;
measuring Raman spectral data in the second region;
delivering a therapeutic agent into the body structure in a selected region with the Raman probe.

48. The method of claim 47 wherein the step of inserting a Raman probe further comprises inserting a needle and measuring the Raman spectral data by detecting light from a region and processing the detected light with a data processor to generate Raman spectral data.

49. The method of claim 47 further comprising comparing the measured Raman spectral data with reference data stored in a memory, performing an epidural injection and comparing measured Raman data from a plurality of tissue layers with stored reference data that is correlated with each tissue layer, the method including inserting a probe with a curved distal end and a side opening for fluid delivery; and performing a loss of resistance method to monitor insertion of the probe into the body structure.
